(19) United States
(12) Patent Application Publication
(10) Pub. No.: US 2011/0092436 A1
(43) Pub. Date: Apr. 21, 2011

(54) COMPOUNDS FOR TREATING SYMPTOMS ASSOCIATED WITH PARKINSON'S DISEASE

(75) Inventors: Markus Mandler, Vienna (AT); Frank Mattner, Vienna (AT); Walter Schmidt, Vienna (AT)

(73) Assignee: AFFIRIS AG, Vienna (AT)

(21) Appl. No.: 12/997,702

(22) PCT Filed: Jun. 12, 2009
(86) PCT No.: PCT/AT2009/000237

§ 371 Date: Dec. 13, 2010

(30) Foreign Application Priority Data
Jun. 12, 2008 (AT) A 951/2008
Jun. 12, 2008 (AT) A 952/2008

(51) Int. Cl. A61K 38/08; A61P 25/16

(52) U.S. Cl. 514/17.7

(57) ABSTRACT

The present invention relates to a compound comprising a peptide for treating, preventing and/or ameliorating motor symptoms of Parkinson's disease, said peptide having a binding capacity to an antibody which is specific for an epitope of the amyloid-beta-peptide (Aβ).

The present invention relates to methods and means for preventing, ameliorating and treat symptoms associated with Parkinson's disease.

Alzheimer's disease (AD) and Parkinson's Disease (PD) are the most common causes of dementia and movement disorders in humans. While AD is characterized by the accumulation of amyloid-beta protein (forming so called Aβ plaques) which is derived from amyloid precursor protein. (APP), PD patients are developing pathologic accumulation of alpha-Synuclein (a-Syn, aSyn; forming so called Lewy Bodies). Both of these molecules are considered to be the major disease causing agents for these neurodegenerative disorders. Both diseases, AD and PD, are associated with degeneration of neurons and synaptic connections, deficiency of specific neurotransmitters, and abnormal accumulation of mis-folded proteins, whose non pathogenic paternal proteins play important roles in normal central nervous system functions.

Recently, a novel form of dementia associated with movement disorders but clinical symptoms differing from those of AD, vascular dementia or idiopathic parkinsonism has been defined clinically. This novel syndrome has been defined as dementia with Lewy bodies or Parkinson's with dementia (DLB/PDD). DLB/PDD is amounting to up to 25% of all dementia cases and has to be considered as second most prominent form of dementia in the elderly. The disease is characterized by the formation of wide-spread Lewy body pathology associated with extensive amyloid deposition. This presence of widespread Lewy bodies differentiates the DLB/PDD cases from all other types of dementia as well as from other movement disorders. The neurological assessment of DLB/PDD shows prominent abnormalities in attention, in executive functions, in memory as well as behavioural and motoric alterations.

It is currently believed that aSyn and Aβ have distinct, as well as convergent, pathogenic effects on the nervous system. Synucleins are believed to affect motoric function more severely than cognitive function, whereas amyloid β peptides are described to have opposite effects.In addition, aSYN and Aβ could interact more directly by engaging synergistic neurodegenerative pathways. It has been recently shown that different pathologic molecules including Aβ, Tau as well as aSyn can mutually exacerbate toxic effects in preclinical disease models and indicate an important function of Aβ in different neurodegenerative conditions. In a recent transgenic animal model for DLB/PDD it has been shown that coexpression of both molecules, haSYN and hAPP, in mice leads to the development of cognitive and motor alterations accompanied by loss of cholinergic neurons and reduction in synaptic vesicles, formation of extensive amyloid plaques, and haSYN-immunoreactive intraneuronal fibrillar inclusions. All of these features are also found in the DLB/PDD syndrome.

Current therapies of symptoms of Parkinson's disease involve the administration of dopaminergic agents to patients suffering from said disease. Dopaminergic agents are believed to reduce the symptoms of Parkinson's disease because it is believed that these symptoms are caused by the deprivation of dopamine in the brain. The insufficiency of dopamine in the brain may therefore be compensated by administering to the patient dopaminergic agents, such as dopamine agonists or dopamine precursors, e.g. levodopa. There is no established cure for Parkinson's disease, which means that the symptoms worsen, necessitating an increase in daily dosage of the medicament as the disease progresses. Furthermore, the chronic use of increased dosages of levodopa leads to the development of motor complications, such as wearing off and involuntary movements (dyskinesia).

The symptoms of motor dysfunction can be improved by levodopa treatment especially combined with other compounds that improve its efficacy.

One of the major disadvantages of the administration of dopaminergic agents is that these agents have to be administered at regular intervals. Furthermore these agents lead only to an increase of dopaminergeic agents in the patient without removing the cause of the symptoms of Parkinson's disease, namely a-Syn plaques.

It is an object of the present invention to provide means for treating symptoms of Parkinson's disease sustainably by reducing the amount of a-Syn deposits.

The present invention relates to a compound comprising a peptide for treating and/or ameliorating motor symptoms of Parkinson's disease, said peptide having a binding capacity to an antibody which is specific for an epitope of the amyloid-beta-peptide (Aβ).

It surprisingly turned out that compounds capable to induce antibodies directed to the amyloid-beta-peptide and, hence, employable to treat beta-amyloidoses such as Alzheimer's disease, can be used to treat and ameliorate the symptoms of Parkinson's disease, in particular the motor symptoms of Parkinson's disease. The antibodies formed by the administration of said compounds reduce surprisingly the amount of a-Syn deposits.

“Motor symptoms”, as used herein, refers to those symptoms of the Parkinson's disease which are described in the EMEA Guideline on Clinical Investigation of Medicinal Products in the Treatment of Parkinson's Disease (CPMP/EWP/563/95 Rev.1) that affect the motor behaviour of a patient suffering from said disease and affects autonomic functions of a patient as well. These symptoms include but are not limited to the core symptoms resting tremor, bradykinesia, rigidity, postural instability as well as stooped posture, dystonia, fatigue, impaired fine motor dexterity and motor coordination, impaired gross motor coordination, poverty of movement (decreased arm swing), akathisia, speech problems, such as softness of voice or slurred speech caused by lack of muscle control, loss of facial expression, or “masking”, micrographia, difficulty swallowing, sexual dysfunction, drooling.

As used herein, the term “epitope” refers to an immunogenic region of an antigen which is recognized by a particular antibody molecule. An antigen may possess one or more epitopes, each capable of binding an antibody that recognizes the particular epitope.

The term “peptide having a binding capacity to an antibody which is specific for an epitope of the amyloid-beta-peptide” means that said peptide can be bound to an amyloid-beta peptide specific antibody which has been produced by the administration of amyloid-beta peptide or fragments thereof to a mammal. Said peptide having said binding capacity is able to induce the formation of amyloid-beta peptide specific antibodies in a mammal. The latter antibodies bind consequently to the compound of the present invention as well as to the amyloid-beta peptide.

According to a preferred embodiment of the present invention said epitope of the amyloid-beta-peptide is selected from the group consisting of DAEFRH, EFRHDSGY, pEFRHDSGY, EVHHQKL, HQKLVF and HQKLVFFAED.

It is particularly preferred to use compounds of the present invention which are able to bind to antibodies directed to/specific for the aforementioned naturally occurring epitopes of the amyloidbeta-peptide. Consequently the compound according to the present invention may comprise a peptide having one of said amino acid sequences.

In another embodiment of the present invention the compound of the present invention does preferably not comprise a peptide having the amino acid sequence DAEFRH, EFRHDSGY, pEFRHDSGY, EVHHQKL, HQKLVF and HQKLVFFAED, but, however, also binds to amyloid-beta-specific antibodies.

For identifying such antibody-inducing peptides phage libraries and peptide libraries can be used. Of course it is also possible to identify such peptides by using means of combinatorial chemistry. All of these methods involve the step of contacting a peptide of a pool of peptides with an amyloid-beta peptide specific antibody. The peptides of the pool binding to said antibody can be isolated and sequenced, if the amino acid sequence of the respective peptide is unknown.

In the following peptides are listed which are able to induce the formation of amyloid-beta antibodies in a mammal. These peptides can also be used for reducing symptoms of Parkinson's disease.

According to a preferred embodiment of the present invention the peptide comprises the amino acid sequence

X₁X₂X₃X₄X₅X₆X₇, (Formula I)

wherein X₁ is G or an amino acid with a hydroxy group or a negatively charged amino acid, preferably glycine (G), glutamic acid (E), tyrosine (Y), serine (S) or aspartic acid (D),

X₂ is a hydrophobic amino acid or a positively charged amino acid, preferably asparagine (N), isoleucine (I), leucine (L), valine (V), lysine (K), tryptophane (W), arginine (R), tyrosine (Y), phenylalanine (F) or alanine (A),

X₃ is a negatively charged amino acid, preferably aspartic acid (D) or glutamic acid (E),

X₄ is an aromatic amino acid or a hydrophobic amino acid or leucine (L), preferably tyrosine (Y), phenylalanine (F) or leucine (L),

X₅ is histidine (H), lysine (K), tyrosine (Y), phenylalanine (F) or arginine (R), preferably histidine (H), phenylalanine (F) or arginine (R), and

X₆ is not present or serine (S), threonine (T), asparagine (N), glutamine (Q), aspartic acid (D), glutamic acid (E), arginine (R), isoleucine (I), lysine (K), tyrosine (Y), or glycine (G), preferably threonine (T), asparagine (N), aspartic acid (D), arginine (R), isoleucine (I) or glycine (G),

X₇ is not present or any amino acid, preferably proline (P), tyrosine (Y), threonine (T), glutamine (Q), alanine (A), histidine (H) or serine (S),

preferably EIDYHR, ELDYHR, EVDYHR, DIDYHR, DLDYHR, DVDYHR, DI-DYRR, DLDYRR, DVDYRR, DKELRI, DWELRI, YREFFI, YREFRI, YAEFRG, EAEFRG, DYEFRG, ELEFRG, DRELRI, DKELKI, DRELKI, GREFRN, EYEFRG, DWEFRDA, SWEFRT, DKELR, SFEFRG, DAEFRWP, DNEFRSP, GSEFRDY, GAEFRFT, SAEFRTQ, SAEFRAT, SWEFRNP, SWEFRLY, SWELRQA, SVEFRYH, SYEFRHH, SQEFRTP, SSEFRVS, DWEFRD, DAELRY, DWELRQ, SLEFRF, GPEFRW, GKEFRT, AYEFRH, DKE(Nle)R, DKE(Nva)R or DKE(Cha)R.

According to a further embodiment of the present invention said peptide comprises the amino acid sequence

X₁RX₂DX₃(X₄)ₙ(X₅)ₘ(X₆)ₒ, (Formula II),

wherein X₁ is isoleucine (I) or valine (V),

X₂ is tryptophan (W) or tyrosine (Y),

X₃ is threonine (T), valine (V), alanine (A), methionine (M), glutamine (Q) or glycine (G),

X₄ is proline (P), alanine (A), tyrosine (Y), serine (S), cysteine (C) or glycine (G),

X₅ is proline (P), leucine (L), glycine (G) or cysteine (C),

X₆ is cysteine (C),

n, m and o are, independently, 0 or 1,

preferably IRWDTP(C), VRWDVYP(C), IRYDAPL(C), IRYDMAG(C), IRWDTSL(C), IRWDQP(C), IRWDG(C) or IRWDGG(C).

The peptide of the compound of the present invention may comprise the amino acid sequence

EX₁WHX₂X₃(X₄)ₙ(X₅)ₘ (Formula III),

wherein X₁ is valine (V), arginine (R) or leucine (L),

X₂ is arginine (R) or glutamic acid (E),

X₃ is alanine (A), histidine (H), lysine (K), leucine (L), tyrosine (Y) or glycine (G),

X₄ is proline (P), histidine (H), phenylalanine (F) or glutamine (Q) or Cysteine

X₅ is cysteine (C),

n and m are, independently, 0 or 1,

preferably EVWHRHQ(C), ERWHEKH(C), EVWHRLQ(C), ELWHRYP(C), ELWHRAF(C), ELWHRA(C), EVWHRG(C), EVWHRH(C) and ERWHEK(C), preferably EVWHRHQ(C), ERWHEKH(C), EVWHRLQ(C), ELWHRYP(C) or ELWHRAF(C).

According to a particularly preferred embodiment of the present invention the peptide comprises the amino acid sequence QDFRHY(C), SEFKHG(C), TSFRHG(C), TSVFRH(C), TPFRHT(C), SQFRHY(C), LMFRHN(C), SAFRHH(C), LPFRHG(C), SHFRHG(C), ILFRHG(C), QFKHDL(C), NWFPHP(C), EEFKYS(C), NELRHST(C), GEMRHQP(C), DTYFPRS(C), VELRHSR(C), YSMRHDA(C), AANYFPR(C), SPNQFRH(C), SSSFFPR(C), EDWFFWH(C), SAGSFRH(C), QVMRHHA(C), SEFSHSS(C), QPNLFYH(C), ELFKHHL(C), TLHEFRH(C), ATFRHSP(C), APMYFPH(C), TYFSHSL(C), HEPLFSH(C), SLMRHSS(C), EFLRHTL(C), ATPLFRH(C), QELKRYY(C), THTDFRH(C), LHIPFRH(C), NELFKHF(C), SQYFPRP(C), DEHPFRH(C), MLPFRHG(C), SAMRHSL(C), TPLMFWH(C), LQFKHST(C), ATFRHST(C), TGLMFKH(C), AEFSHWH(C), QSEFKHW(C), AEFMHSV(C), ADHDFRH(C), DGLLFKH(C), IGFRHDS(C), SNSEFRR(C), SELRHST(C), THMEFRR(C), EELRHSV(C), QLFKHSP(C), YEFRHAQ(C), SNFRHSV(C), APIQFRH(C), AYFPHTS(C), NSSELRH(C), TEFRHKA(C), TSTEMWH(C), SQSYFKH(C), (C)SEFKH, SEFKH(C), (C)HEFRH or HEFRH(C).

According to another preferred embodiment of the present invention the peptide comprises the amino acid sequence

(X₁)ₘGX₂X₃X₄FX₅X₆(X₇)ₙ (Formula IV),

wherein X₁ is serine (S), alanine (A) or cysteine (c),

X₂ is serine (S), threonine (T), glutamic acid (E), aspartic acid (D), glutamine (Q) or methionine (M),

X₃ is isoleucine (I), tyrosine (Y), methionine (M) or leucine (L),

X₄ is leucine (L), arginine (R), glutamine (Q), tryptophan (W), valine (V), histidine (H), tyrosine (Y), isoleucine (I), lysine (K) methionine (M) or phenylalanine (F),

X₅ is alanine (A), phenylalanine (F), histidine (H), asparagine (N), arginine (R), glutamic acid (E), isoleucine (I), glutamine (Q), aspartic acid (D), proline (P) or tryptophane (W), glycine (G)

X₆ is any amino acid residue,

X₇ is cysteine (C),

m and n are, independently, 0 or 1,

preferably SGEYVFH(C), SGQLKFP(C), SGQIWFR(C), SGEIHFN(C), GQIWFIS(C), GQIIFQS(C), GQIRFDH(C), GEMWFAL(C), GELQFPP(C), GELWFP(C), GEMQFFI(C), GELYFRA(C), GEIRFAL(C), GMIVFPH(C), GEIWFEG(C), GDLKFPL(C), GQILFPV(C), GELFFPK(C), GQIMFPR(C), GSLFFWP(C), GEILFGM(C), GQLKFPF(C), GTIFFRD(C), GQIKFAQ(C), GTLIFHH(C), GEIRFGS(C), GQIQFPL(C), GEIKFDH(C), GEIQFGA(C), GELFFEK(C), GEIRFEL(C), GEIYFER(C), SGEIYFER(C), AGEIYFER(C) or (C)GEIYFER.

According to a further preferred embodiment of the present invention the peptide comprises the amino acid sequence

(X₁)ₘHX₂X₃X₄X₅FX₆(X₇)ₙ (Formula V),

wherein X₁ is serine (S), threonine (T) or cysteine (C),

X₂ is glutamine (Q), threonine (T) or methionine (M),

X₃ is lysine (K) or arginine (R),

X₄ is leucine (L), methionine (M),

X₅ is tryptophane (W), tyrosine (Y), phenylalanine (F) or isoleucine (I),

X₆ is asparagine (N), glutamic acid (E), alanine (A) or cysteine (C),

X₇ is cysteine (C),

n and m are, independently, 0 or 1,

preferably SHTRLYF(C), HMRLFFN(C), SHQRLWF(C), HQKMIFA(C), HMRMYFE(C), THQRLWF(C) or HQKMIF(C).

According to a preferred embodiment of the present invention the peptide comprises the amino acid sequence AIPLFVM(C), KLPLFVM(C), QLPLFVL(C) or NDAKIVF(C).

The compound according to the present invention is preferably a polypeptide/peptide and comprises 4 to 30 amino acid residues, preferably 5 to 25 amino acid residues, more preferably 5 to 20 amino acid residues.

The compound of the present invention may also be part of a polypeptide comprising 4 to 30 amino acid residues.

The peptides exhibiting an affinity to amyloid-beta antibodies may be considered as mimotopes. According to the present invention the term “mimotope” refers to a molecule which has a conformation that has a topology equivalent to the epitope of which it is a mimic. The mimotope binds to the same antigen-binding region of an antibody which binds immunospecifically to a desired antigen. The mimotope will elicit an immunological response in a host that is reactive to the antigen to which it is a mimic. The mimotope may also act as a competitor for the epitope of which it is a mimic in in vitro inhibition assays (e.g. ELISA inhibition assays) which involve the epitope and an antibody binding to said epitope. However, a mimotope of the present invention may not necessarily prevent or compete with the binding of the epitope of which it is a mimic in an in vitro inhibition assay although it is capable to induce a specific immune response when administered to a mammal. The compounds of the present invention comprising such mimotopes (also those listed above) have the advantage to avoid the formation of autoreactive T-cells, since the peptides of the compounds have an amino acid sequence which varies from those of naturally occurring amyloid-beta peptide.

The mimotopes/peptides of the present invention can be synthetically produced by chemical synthesis methods which are well known in the art, either as an isolated peptide or as a part of another peptide or polypeptide. Alternatively, the peptide mimotope can be produced in a microorganism which produces the peptide mimotope which is then isolated and if desired, further purified. The peptide mimotope can be produced in microorganisms such as bacteria, yeast or fungi, in eukaryote cells such as a mammalian or an insect cell, or in a recombinant virus vector such as adenovirus, poxvirus, herpesvirus, Simliki forest virus, baculovirus, bacteriophage, sindbis virus or sendai virus. Suitable bacteria for producing the peptide mimotope include E. coli, B. subtilis or any other bacterium that is capable of expressing peptides such as the peptide mimotope. Suitable yeast types for expressing the peptide mimotope include Saccharomyces cerevisiae, Schizosaccharomyces pombe, Candida, Pichia pastoris or any other yeast capable of expressing peptides. Corresponding methods are well known in the art. Also methods for isolating and purifying recombinantly produced peptides are well known in the art and include e.g. as gel filtration, affinity chromatography, ion exchange chromatography etc.

To facilitate isolation of the peptide mimotope, a fusion polypeptide may be made wherein the peptide mimotope is translationally fused (covalently linked) to a heterologous polypeptide which enables isolation by affinity chromatography. Typical heterologous polypeptides are His-Tag (e.g. His₆; 6 histidine residues), GST-Tag (Glutathione-S-transferase) etc. The fusion polypeptide facilitates not only the purification of the mimotopes but can also prevent the mimotope polypeptide from being degraded during purification. If it is desired to remove the heterologous polypeptide after purification the fusion polypeptide may comprise a cleavage site at the junction between the peptide mimotope and the heterologous polypeptide. The cleavage site consists of an amino acid sequence that is cleaved with an enzyme specific for the amino acid sequence at the site (e.g. proteases).

The mimotopes of the present invention may also be modified at or nearby their N- and/or C-termini so that at said positions a cysteine residue is bound thereto. In a preferred embodiment terminally positioned (located at the N- and C-termini of the peptide) cysteine residues are used to cyclize the peptides through a disulfide bond.

The mimotopes of the present invention may also be used in various assays and kits, in particular in immunological assays and kits. Therefore, it is particularly preferred that the mimotope may be part of another peptide or polypeptide, particularly an enzyme which is used as a reporter in immunological assays. Such reporter enzymes include e.g. alkaline phosphatase or horseradish peroxidase.

The mimotopes according to the present invention preferably are antigenic polypeptides which in their amino acid sequence vary from the amino acid sequence of Aβ or of fragments of Aβ. In this respect, the inventive mimotopes may not only comprise amino acid substitutions of one or more naturally occurring amino acid residues but also of one or more non-natural amino acids (i.e. not from the 20 “classical” amino acids) or they may be completely assembled of such non-natural amino acids. Moreover, the inventive antigens which induce antibodies directed and binding to Aβ1-40/42, AβpE3-40/42, Aβ3-40/42, Aβ11-40/42, AβpE11-40/42 and Aβ14-40/42 (and other N-terminally truncated forms of Aβ starting from amino acid positions 2, 3, 4, 5, 6, 7, 8, 9, 10, 11, 12 and 13) may be assembled of D- or L-amino acids or of combinations of DL-amino acids and, optionally, they may have been changed by further modifications, ring closures or derivatizations. Suitable antibody-inducing antigens may be provided from commercially available peptide libraries. Preferably, these peptides are at least 7 amino acids, and preferred lengths may be up to 16, preferably up to 14 or 20 amino acids (e.g. 5 to 16 amino acid residues). According to the invention, however, also longer peptides may very well be employed as antibody-inducing antigens. Furthermore the mimotopes of the present invention may also be part of a polypeptide and consequently comprising at their N- and/or C-terminus at least one further amino acid residue.

For preparing the mimotopes of the present invention (i.e. the antibody-inducing antigens disclosed herein), of course also phage libraries, peptide libraries are suitable, for instance produced by means of combinatorial chemistry or obtained by means of high throughput screening techniques for the most varying structures (Display: A Laboratory Manual by Carlos F. Barbas (Editor), et al.; Willats WG Phage display: practicalities and prospects. Plant Mol. Biol. 2002 December; 50(6):837-54).

Furthermore, according to the invention also anti-Aβ1-40/42, -AβpE3-40/42-, -Aβ3-40/42-, -Aβ11-40/42- AβpE11-40/42- and Aβ14-40/42-antibody-inducing antigens based on nucleic acids (“aptamers”) may be employed, and these, too, may be found with the most varying (oligonucleotide) libraries (e.g. with 2-180 nucleic acid residues) (e.g. Burgstaller et al., Curr. Opin. Drug Discov. Dev. 5(5) (2002), 690-700; Famulok et al., Acc. Chem. Res. 33 (2000), 591-599; Mayer et al., PNAS 98 (2001), 4961-4965, etc.). In antibody-inducing antigens based on nucleic acids, the nucleic acid backbone can be provided e.g. by the natural phosphor-diester compounds, or also by phosphorotioates or combinations or chemical variations (e.g. as PNA), wherein as bases, according to the invention primarily U, T, A, C, G, H and mC can be employed. The 2′-residues of the nucleotides which can be used according to the present invention preferably are H, OH, F, Cl, NH₂, O-methyl, O-ethyl, O-propyl or O-butyl, wherein the nucleic acids may also be differently modified, i.e. for instance with protective groups, as they are commonly employed in oligonucleotide synthesis. Thus, aptamer-based antibody-inducing antigens are also preferred antibody-inducing antigens within the scope of the present invention.

According to a preferred embodiment of the present invention the compound is coupled to a pharmaceutically acceptable carrier, preferably KLH (Keyhole Limpet Hemocyanin), tetanus toxoid, albumin-binding protein, bovine serum albumin, a dendrimer (MAP; Biol. Chem. 358: 581), peptide linkers (or flanking regions) as well as the adjuvant substances described in Singh et al., Nat. Biotech. 17 (1999), 1075-1081 (in particular those in Table 1 of that document), and O'Hagan et al., Nature Reviews, Drug Discovery 2 (9) (2003), 727-735 (in particular the endogenous immuno-potentiating compounds and delivery systems described therein), or mixtures thereof. The conjugation chemistry (e.g. via heterobifunctional compounds such as GMBS and of course also others as described in “Bioconjugate Techniques”, Greg T. Hermanson) in this context can be selected from reactions known to the skilled man in the art. Moreover, the vaccine composition may be formulated with an adjuvant, preferably a low soluble aluminium composition, in particular aluminium hydroxide. Of course, also adjuvants like MF59 aluminium phosphate, calcium phosphate, cytokines (e.g., IL-2, IL-12, GM-CSF), saponins (e.g., QS21), MDP derivatives, CpG oligos, LPS, MPL, polyphosphazenes, emulsions (e.g., Freund's, SAF), liposomes, virosomes, iscoms, cochleates, PLG microparticles, poloxamer particles, virus-like particles, heat-labile enterotoxin (LT), cholera toxin (CT), mutant toxins (e.g., LTK63 and LTR72), microparticles and/or polymerized liposomes may be used.

The compound of the present invention is preferably bound to the carrier or adjuvant via a linker, which is selected from the group consisting of NHS-poly (ethylene oxide) (PEO) (e.g. NHS-PEO₄-maleimide).

A vaccine which comprises the present compound (mimotope, peptide) and the pharmaceutically acceptable carrier may be administered by any suitable mode of application, e.g. i.d., i.v., i.p., i.m., intranasally, orally, subcutaneously, etc. and in any suitable delivery device (O'Hagan et al., Nature Reviews, Drug Discovery 2 (9), (2003), 727-735). The compound of the present invention is preferably formulated for intravenous, subcutaneous, intradermal or intramuscular administration (see e.g. “Handbook of Pharmaceutical Manufacturing Formulations”, Sarfaraz Niazi, CRC Press Inc, 2004).

The medicament (vaccine) according to the present invention contains the compound according to the invention in an amount of from 0.1 ng to 10 mg, preferably 10 ng to 1 mg, in particular 100 ng to 100 μg, or, alternatively, e.g. 100 fmol to 10 μmol, preferably 10 pmol to 1 μmol, in particular 100 pmol to 100 nmol. Typically, the vaccine may also contain auxiliary substances, e.g. buffers, stabilizers etc.

According to a preferred embodiment of the present invention the motor symptoms of Parkinson's disease are selected from the group consisting of resting tremor, Bradykinesia, rigidity, postural instability, stooped posture, dystonia, fatigue, impaired fine motor dexterity and motor coordination, impaired gross motor coordination, poverty of movement (decreased arm swing), akathisia, speech problems, loss of facial expression, micrographia, difficulty swallowing, sexual dysfunction and drooling.

Another aspect of the present invention relates to the use of a compound according to the present invention for the manufacture of a medicament for treating, preventing and/or ameliorating motor symptoms of Parkinson's disease.

Yet another aspect of the present invention relates to a method for treating and/or ameliorating symptoms, in particular motor symptoms, of Parkinson's disease.

The present invention is further illustrated in the following figures and examples, however, without being restricted thereto.

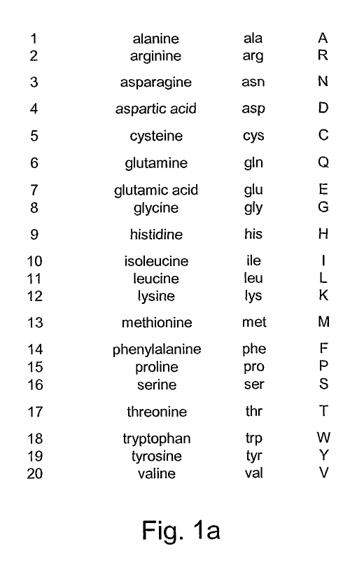

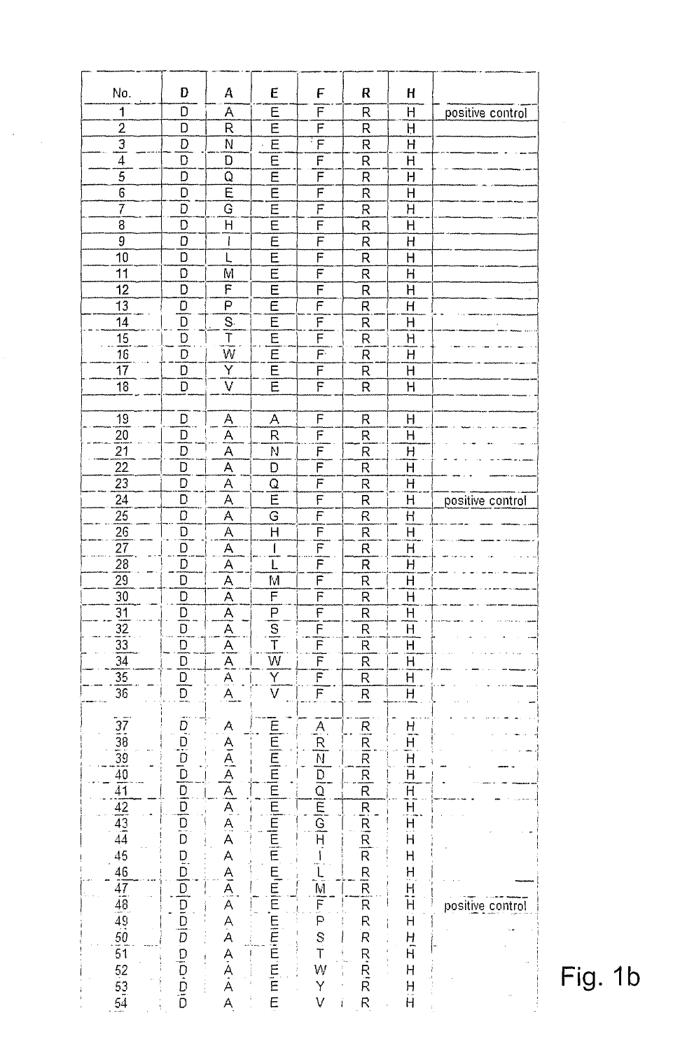

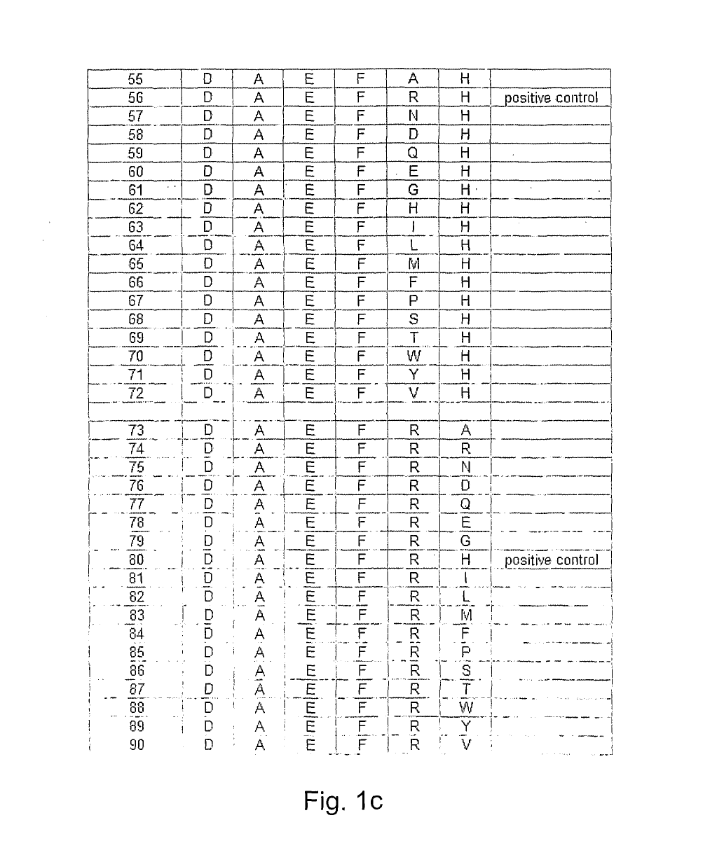

FIG. 1 shows the individualised peptide members of library 4 used for the present screening process.

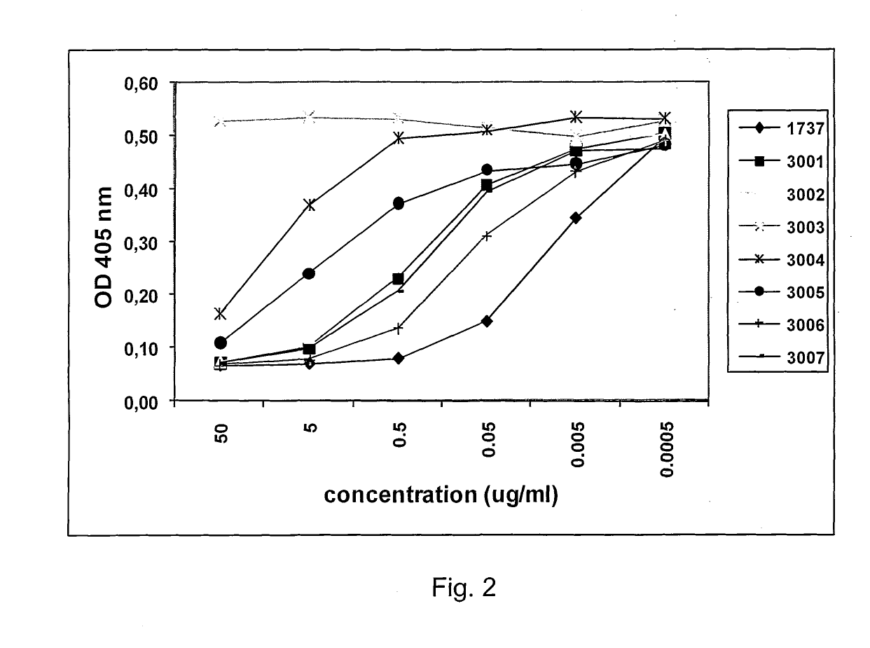

FIG. 2 shows an inhibition assay with mimotopes for DAEFRH.

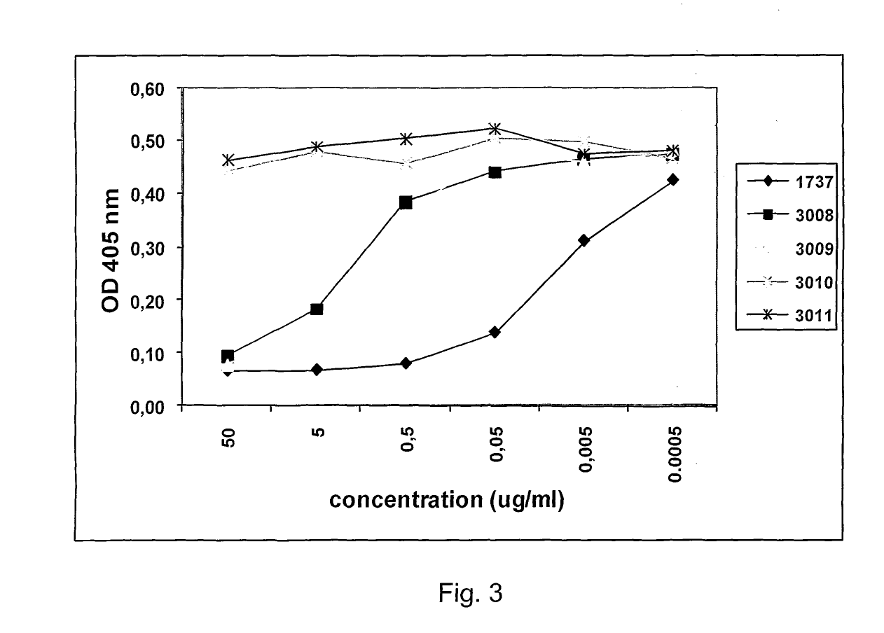

FIG. 3 shows another inhibition assay with other mimotopes for DAEFRH.

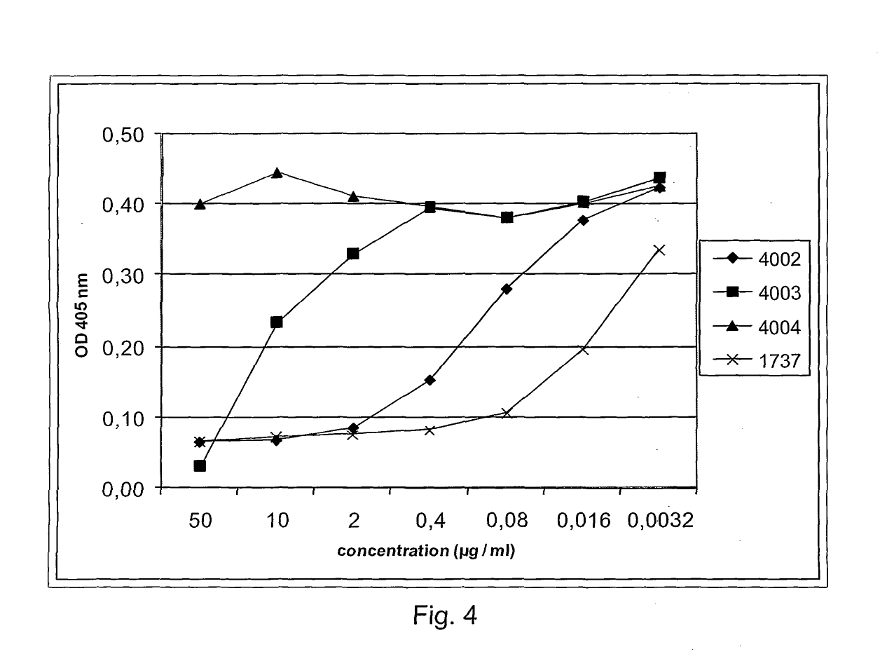

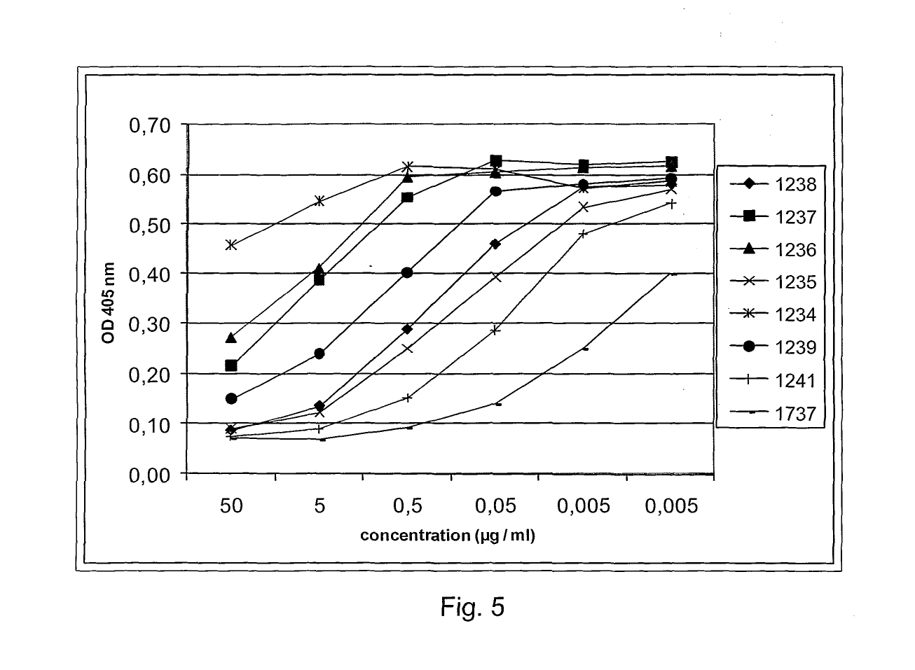

FIGS. 4 and 5 describe the results of inhibition assays performed with mimotope peptides according to the present invention.

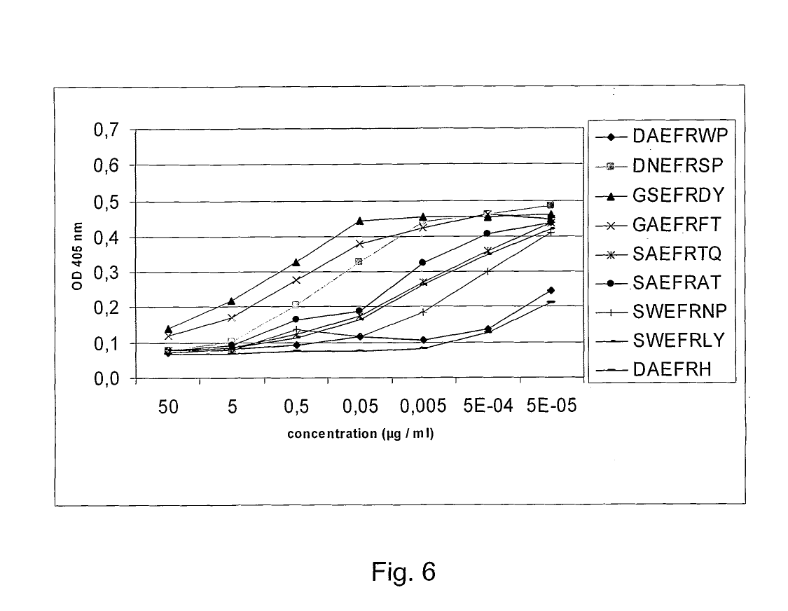

FIGS. 6 to 9 show the results of inhibition assays performed with mimotope peptides 4011-4018, 4019-4025, 4031-4038 and 4061-4064, respectively.

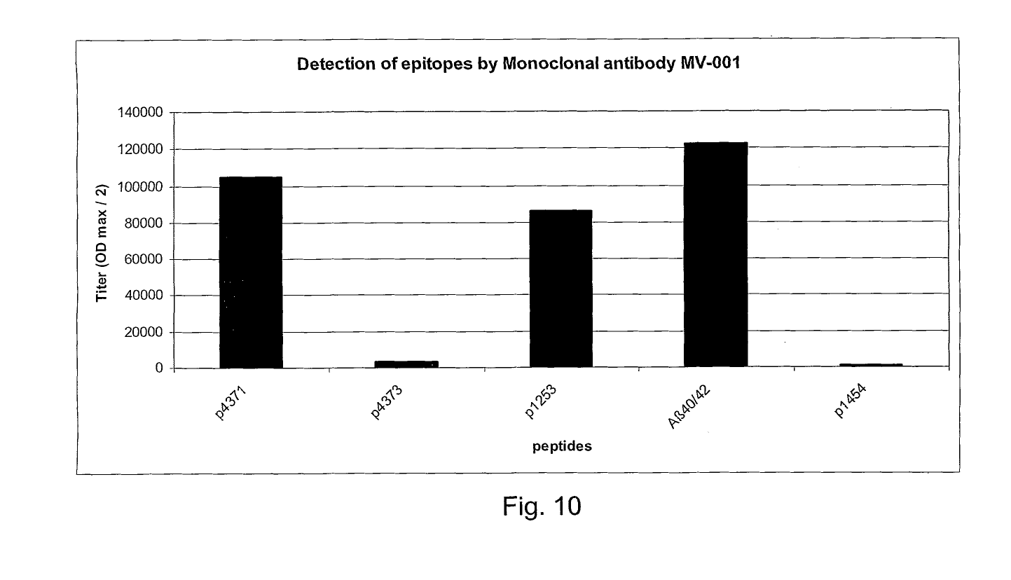

FIG. 10 shows binding of monoclonal antibody MV-001 to specific peptides and recombinant proteins;

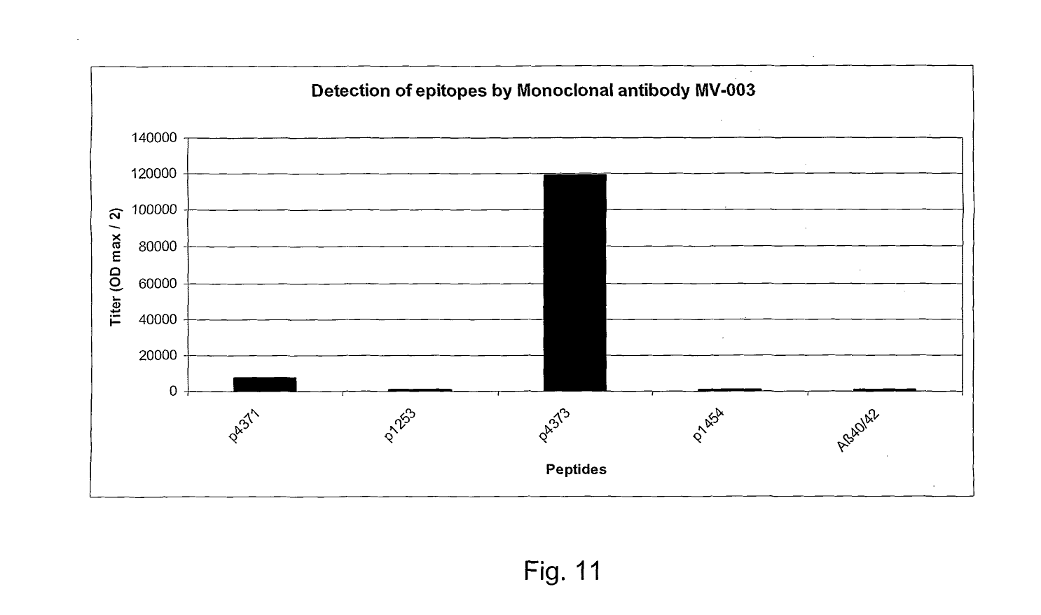

FIG. 11 shows binding of monoclonal antibody MV-003 to specific peptides and recombinant proteins;

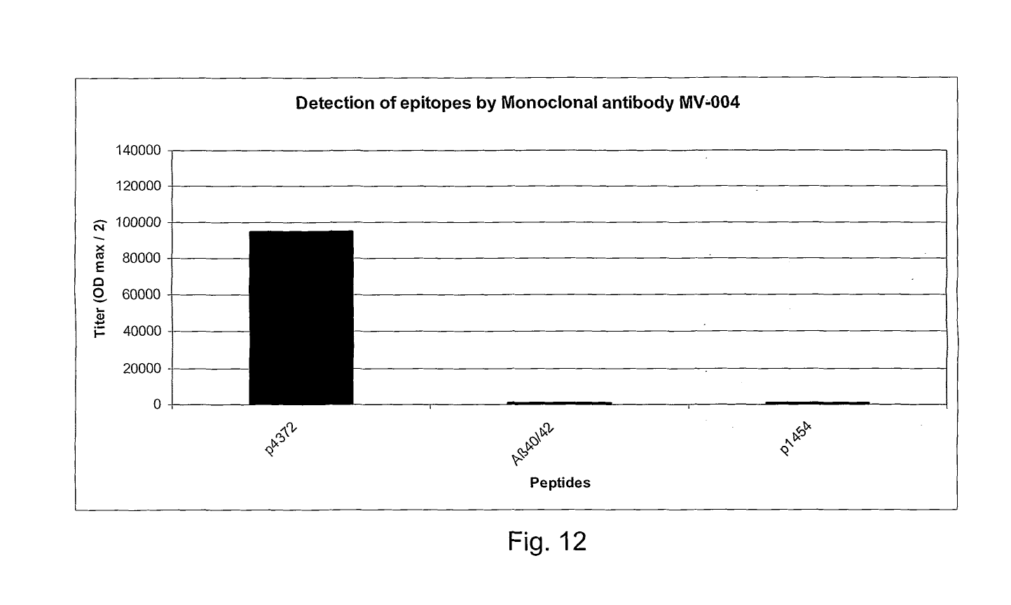

FIG. 12 shows binding of monoclonal antibody MV-004 to specific peptides and recombinant proteins;

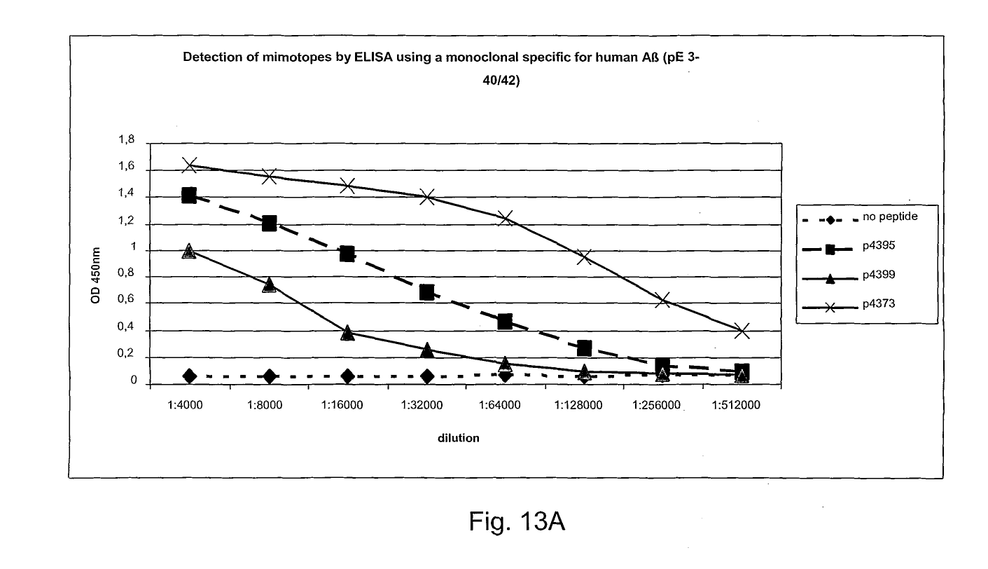

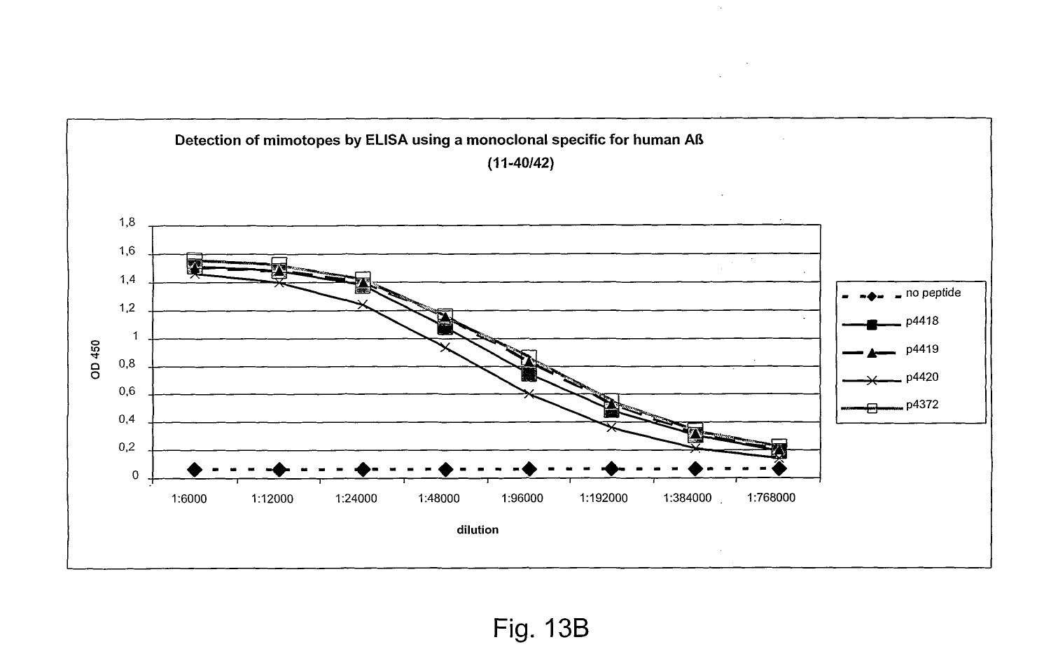

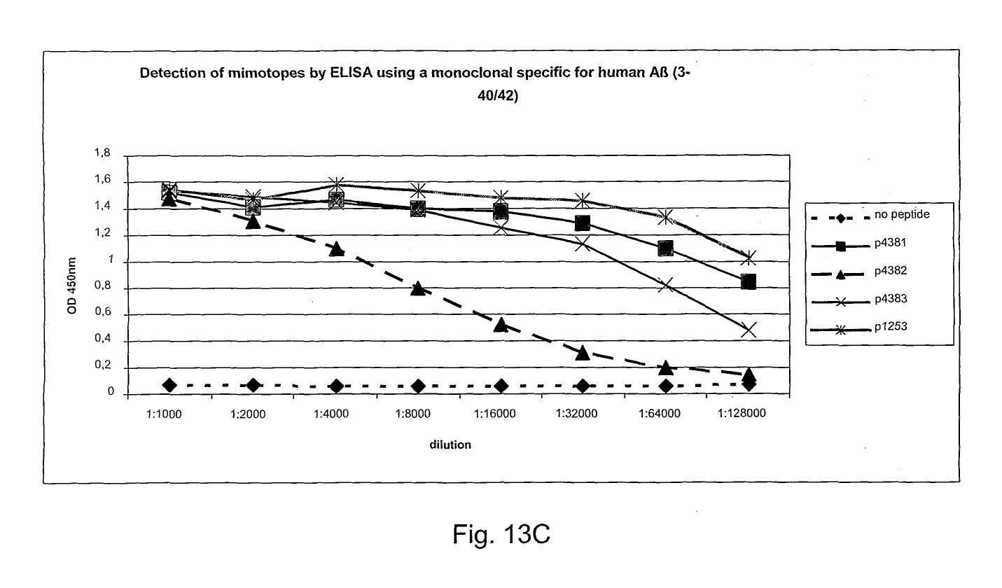

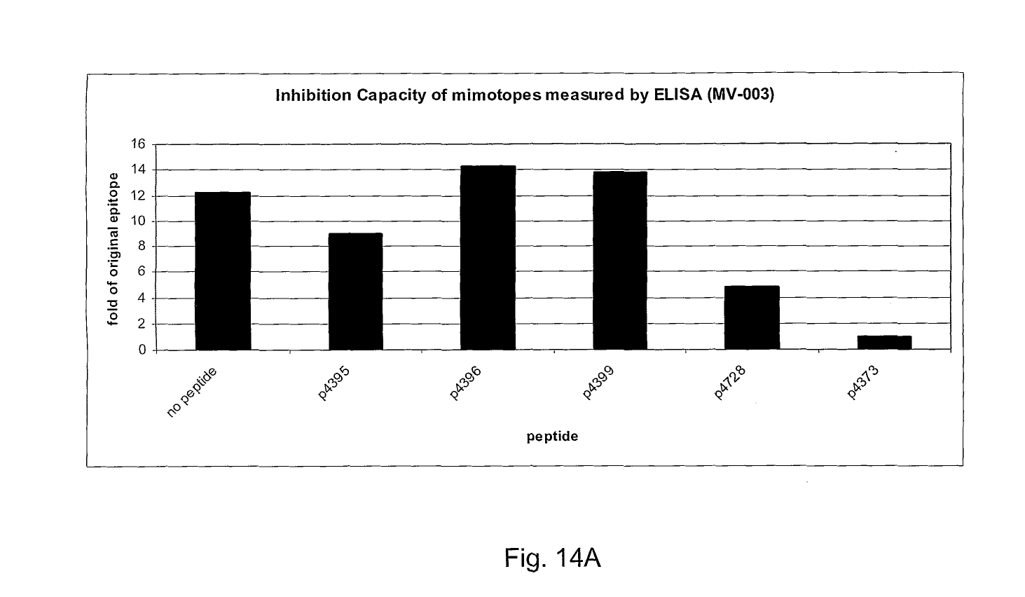

FIG. 13 shows typical binding assays with mimotopes for β-amyloid and N-terminally truncated and/or posttranslationally modified β-amyloid fragments;

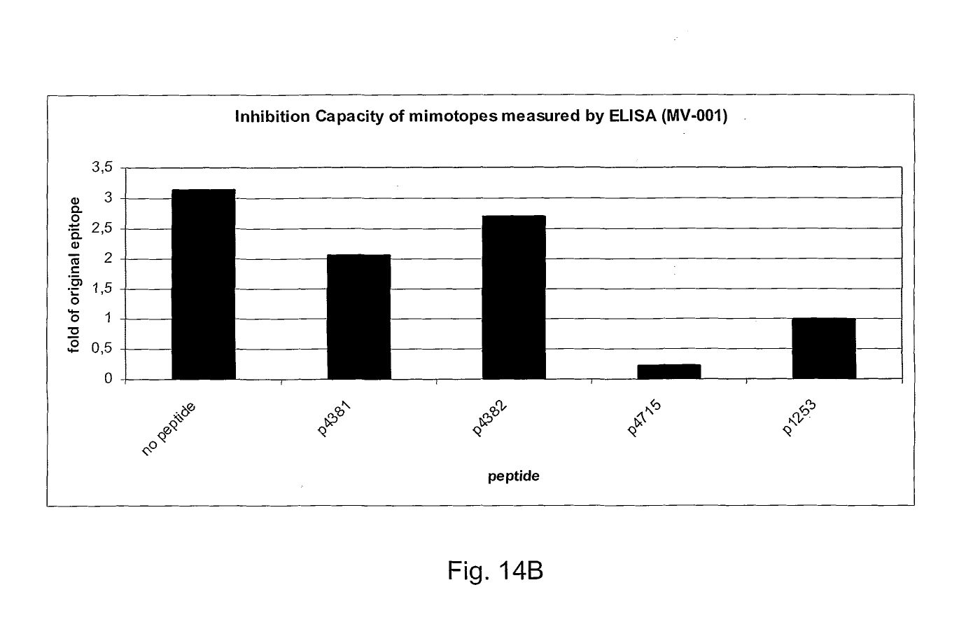

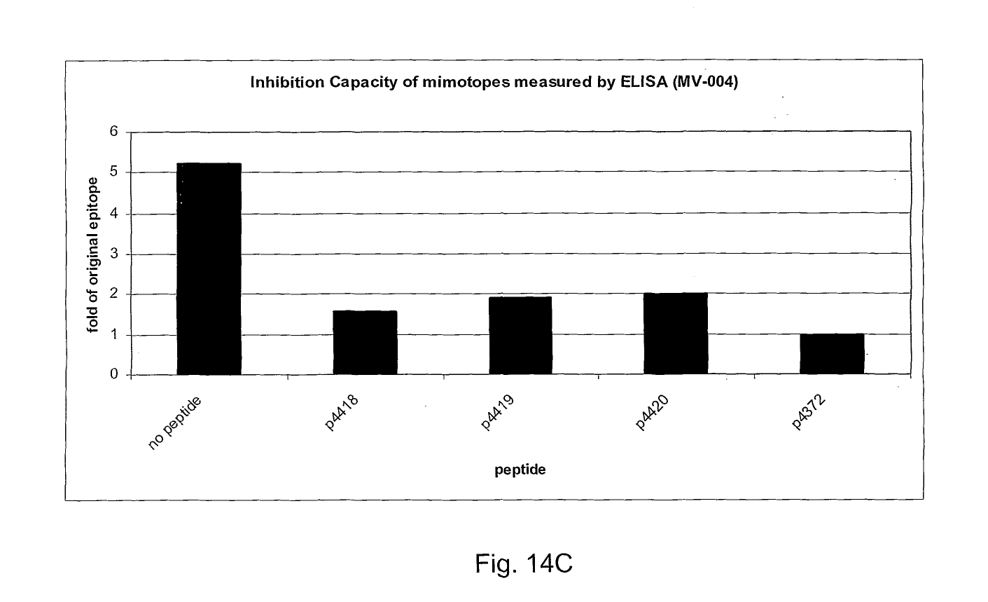

FIG. 14 shows typical inhibition assays with mimotopes for β-amyloid and N-terminally truncated and/or posttranslationally modified β-amyloid fragments;

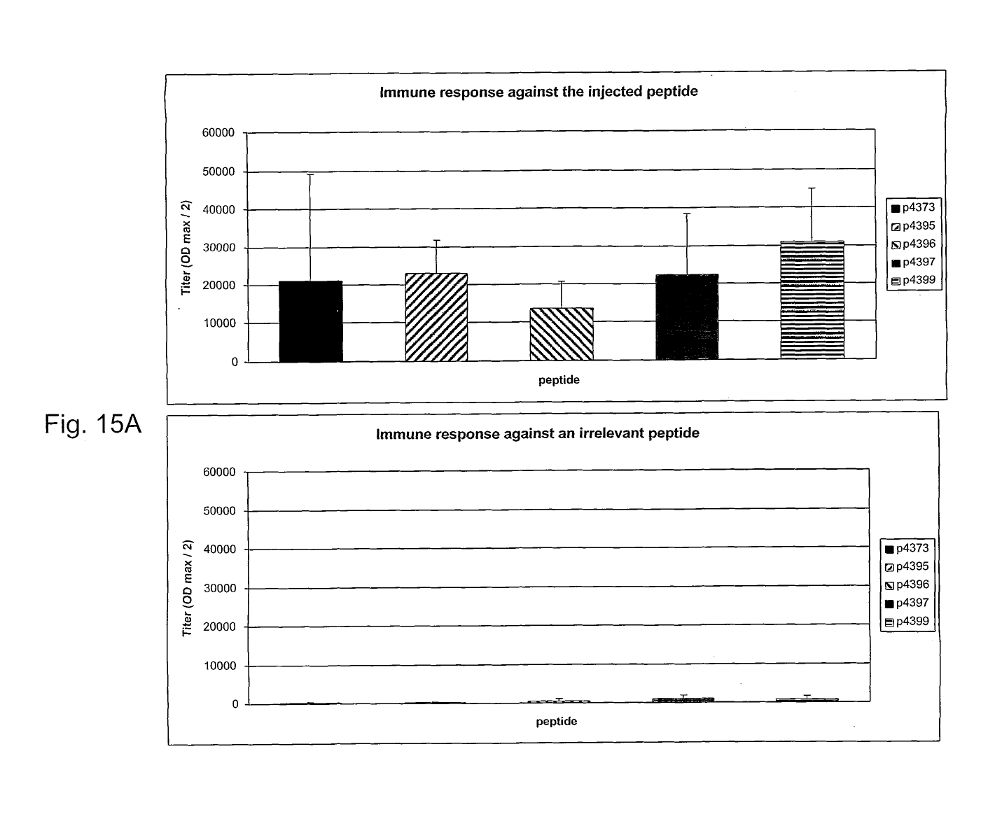

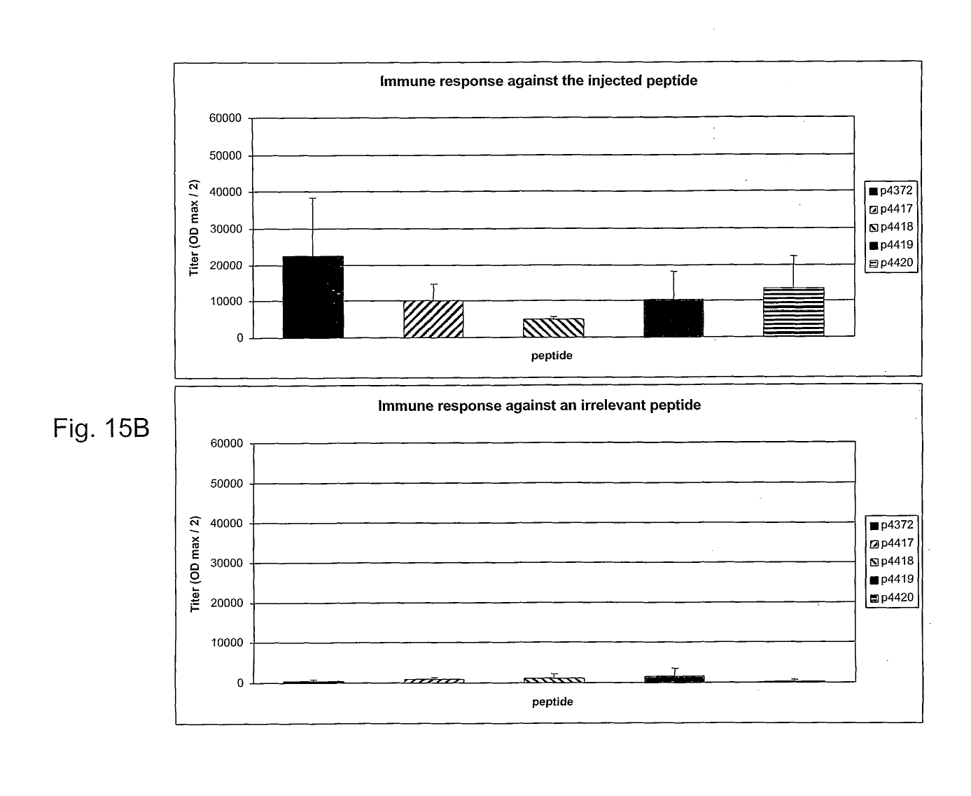

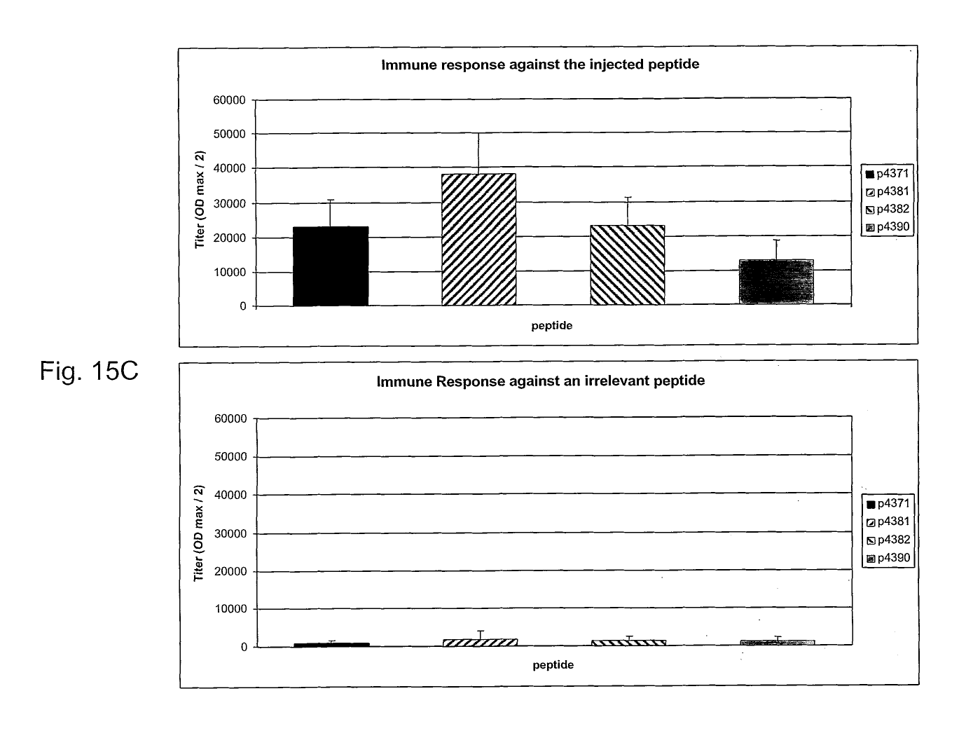

FIG. 15 shows examples for in vivo characterisations of the immune response elicited by mimotope vaccination (injected peptide/irrelevant peptide);

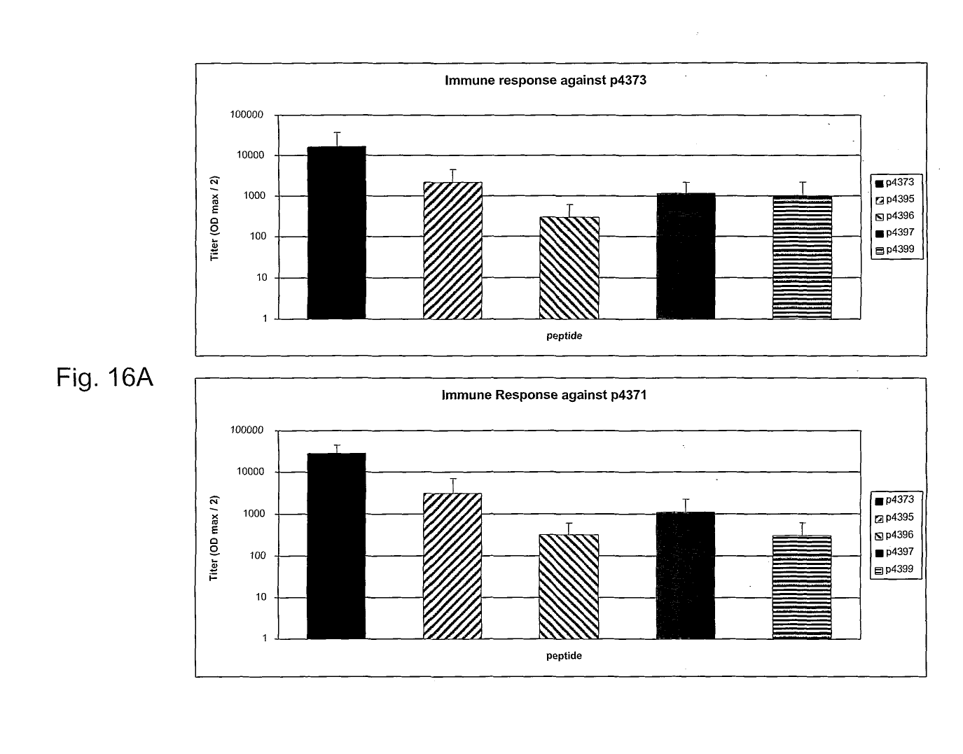

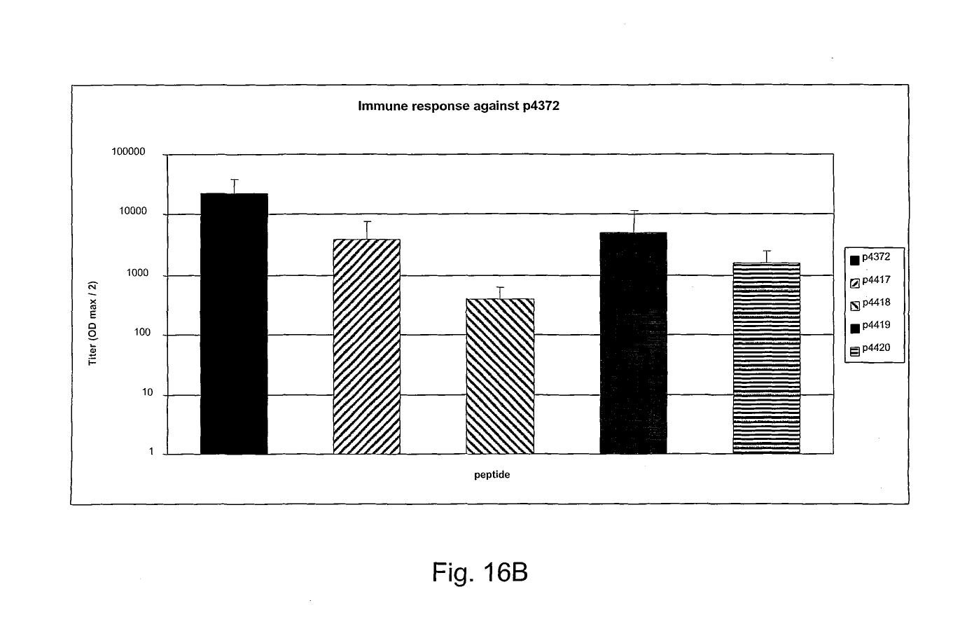

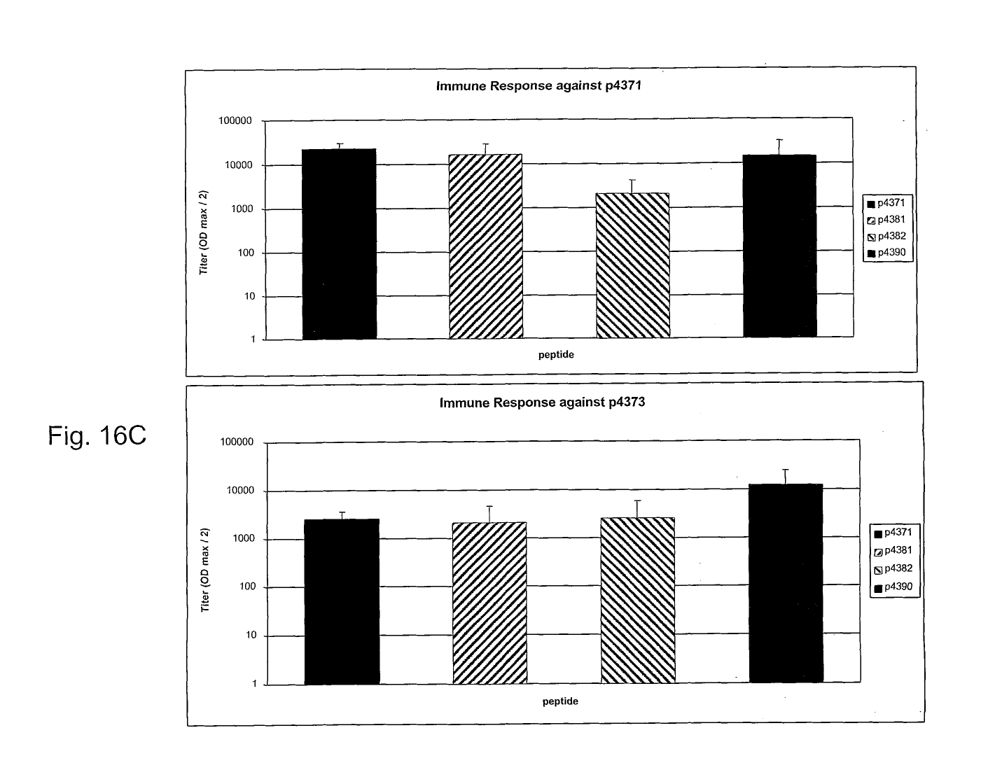

FIG. 16 shows examples for in vivo characterisation of the immune response elicited by mimotope vaccination against Amyloid Beta fragments;

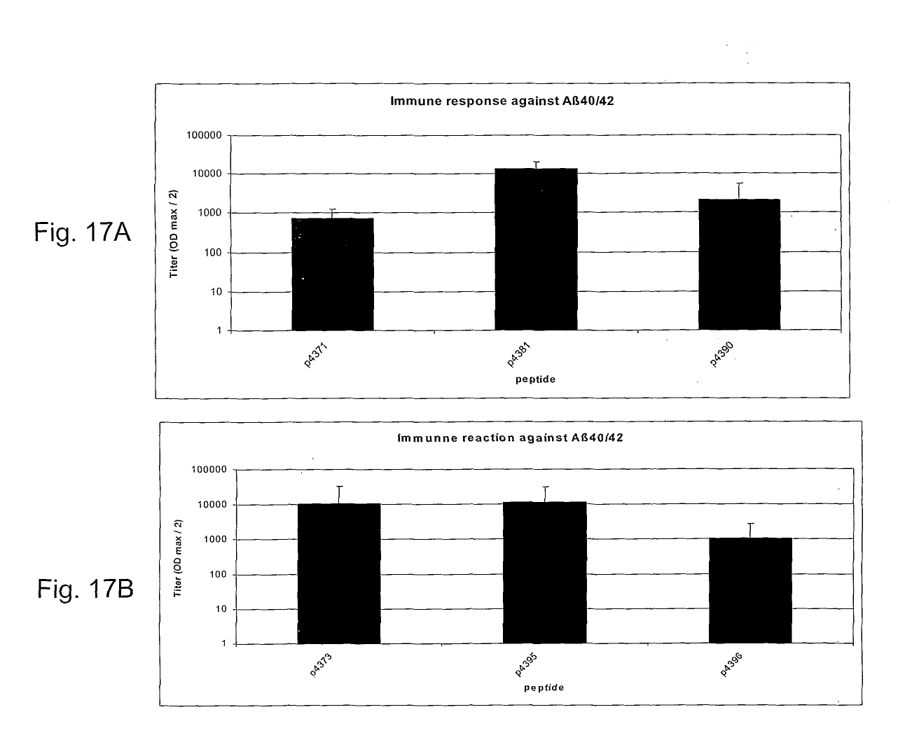

FIG. 17 shows examples for in vivo characterisation of the immune response elicited by mimotope vaccination against full length

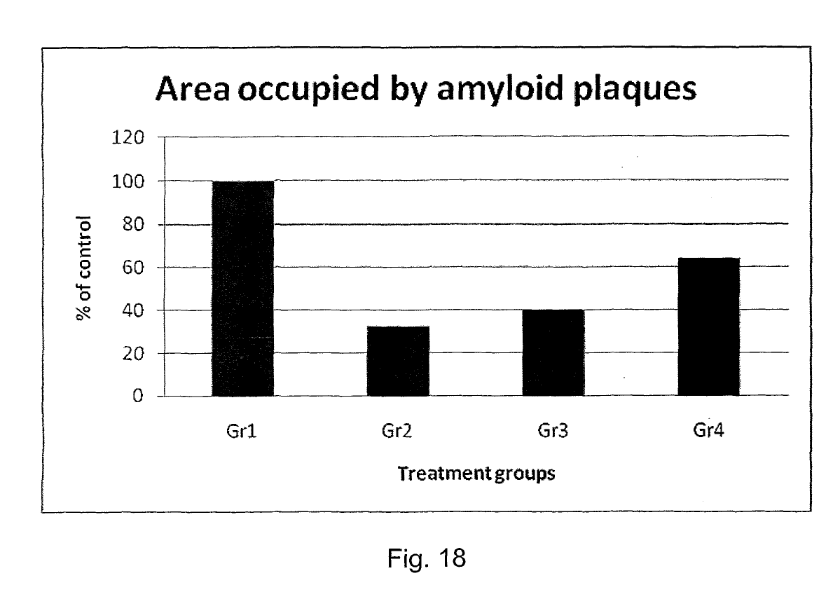

FIG. 18 shows areas occupied by amyloid plaques. Tg2576 were injected 6 times with mimotope vaccines adjuvanted with aluminium hydroxide (ALUM) by s.c. inoculation at monthly intervals. Control mice received PBS-ALUM only. Area occupied by amyloid plaques shown as percent of the control group. Gr1 . . . control group; Gr2 . . . received p4381; Gr3 . . . received p4390; Gr4 . . . received p4715

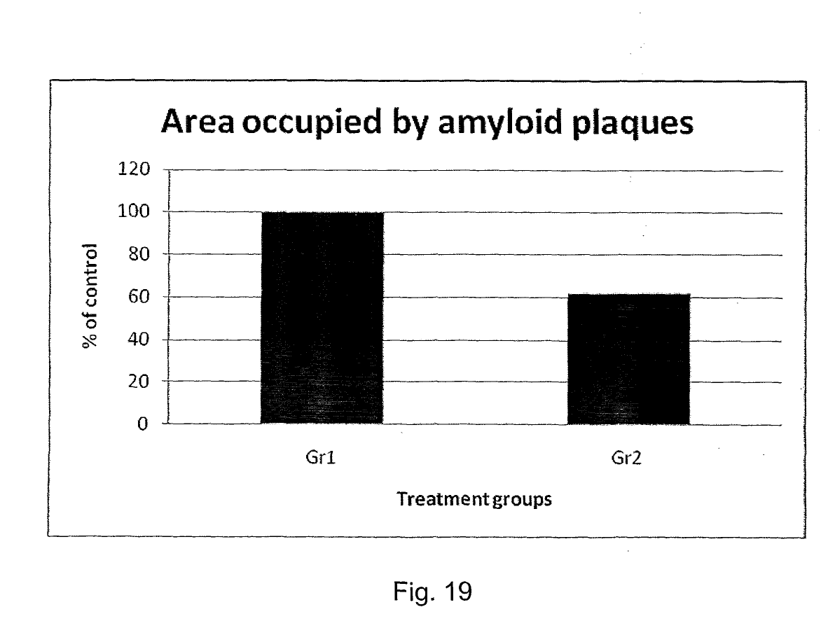

FIG. 19 shows areas occupied by amyloid plaques. Tg2576 were injected 6 times with AFFITOPE vaccines adjuvanted with aluminium hydroxide (ALUM) by s.c. inoculation at monthly intervals. Control mice received PBS-ALUM only. Area occupied by amyloid plaques shown as percent of the control group. Gr1 . . . control group; Gr2 . . . received p4395.

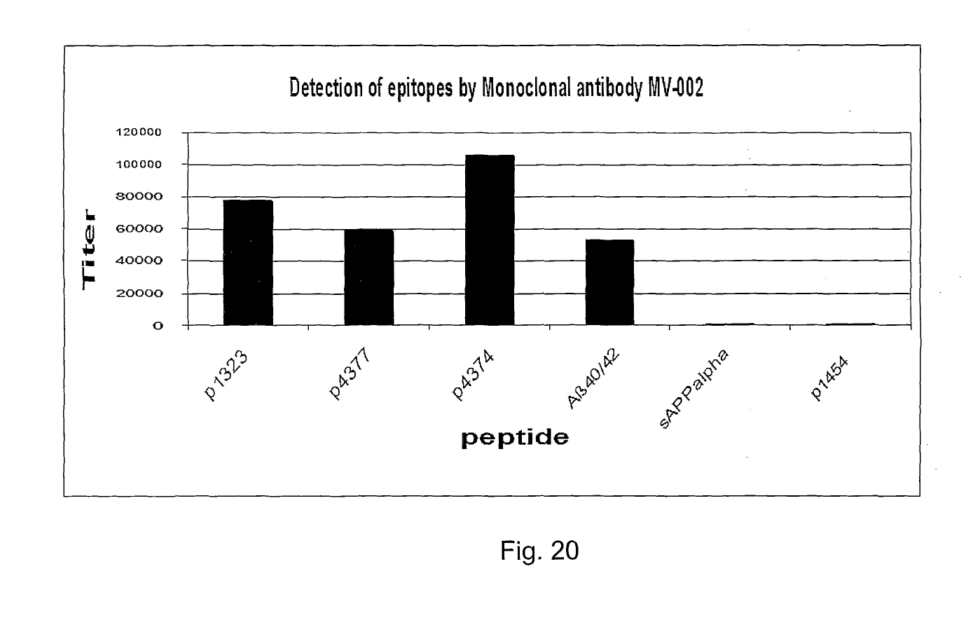

FIG. 20 shows binding of monoclonal antibody MV-002 to specific peptides and recombinant proteins.

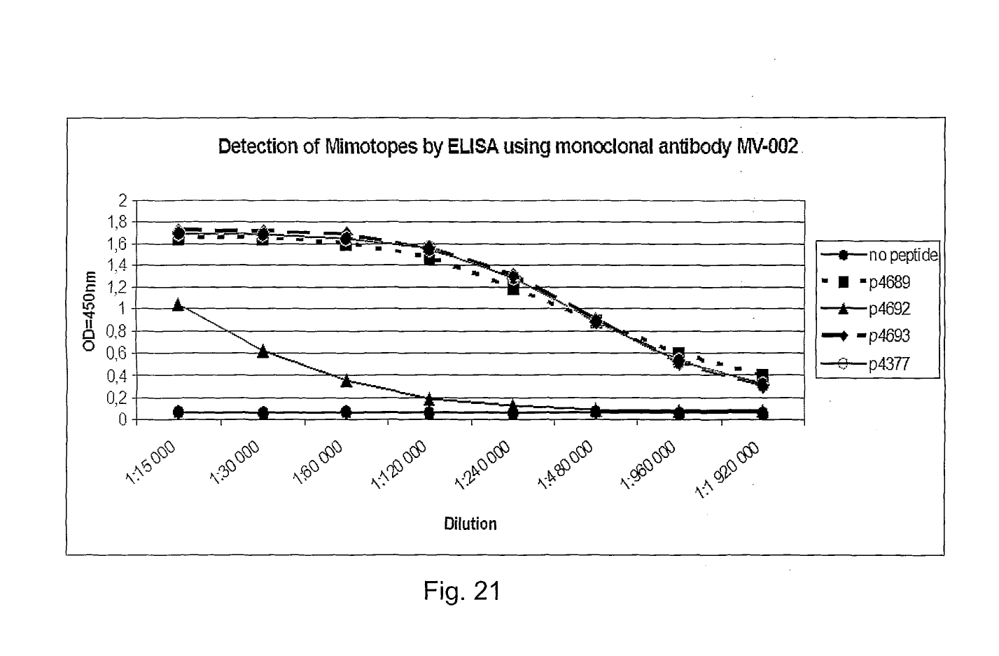

FIG. 21 shows typical binding assays with mimotopes for β-amyloid and N-terminally truncated and/or posttranslationally modified β-amyloid fragments.

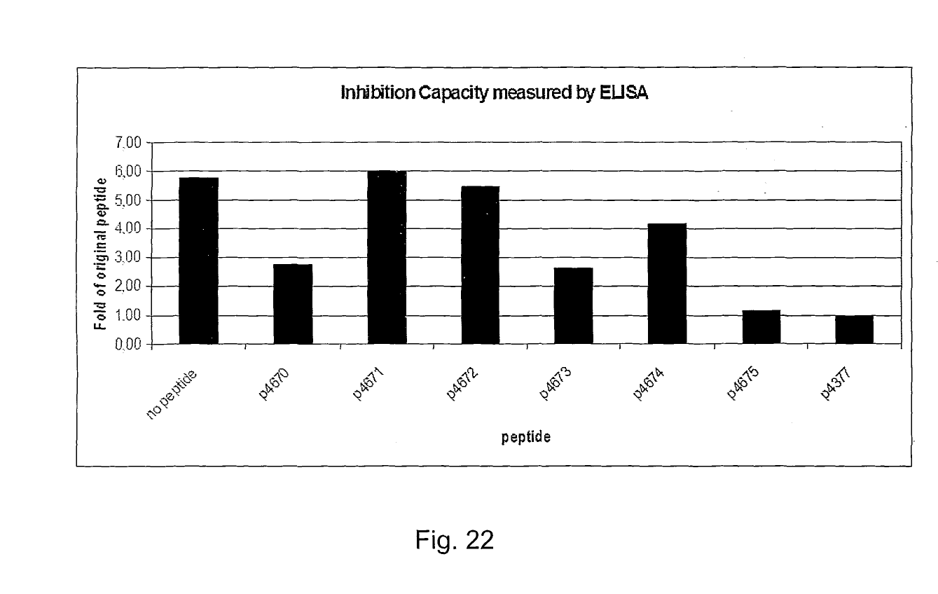

FIG. 22 shows typical inhibition assays with mimotopes for β-amyloid and N-terminally truncated and/or posttranslationally modified β-amyloid fragments.

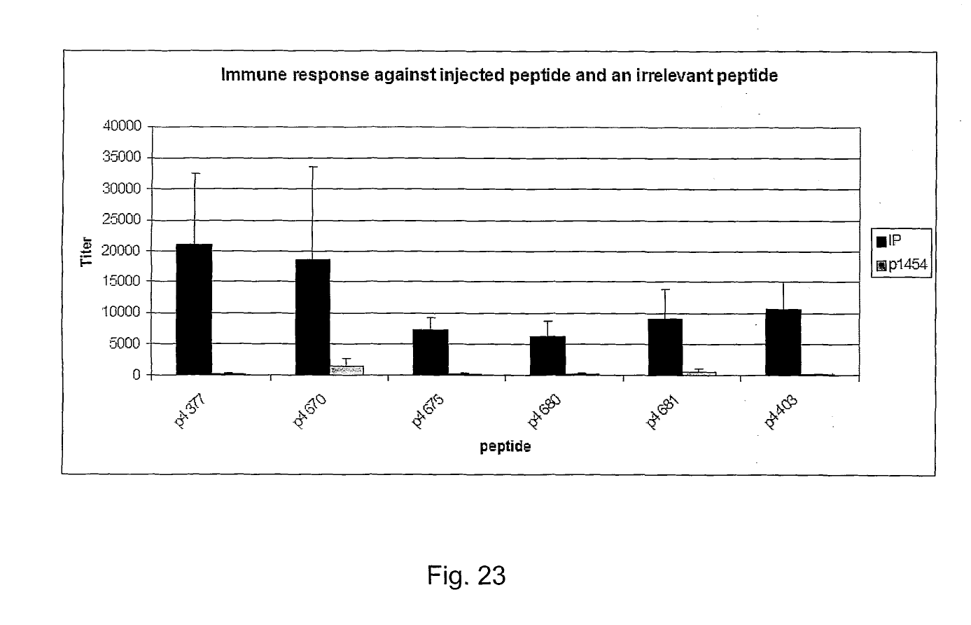

FIG. 23 shows examples for in vivo characterisations of the immune response elicited by mimotope vaccination (injected peptide/irrelevant peptide).

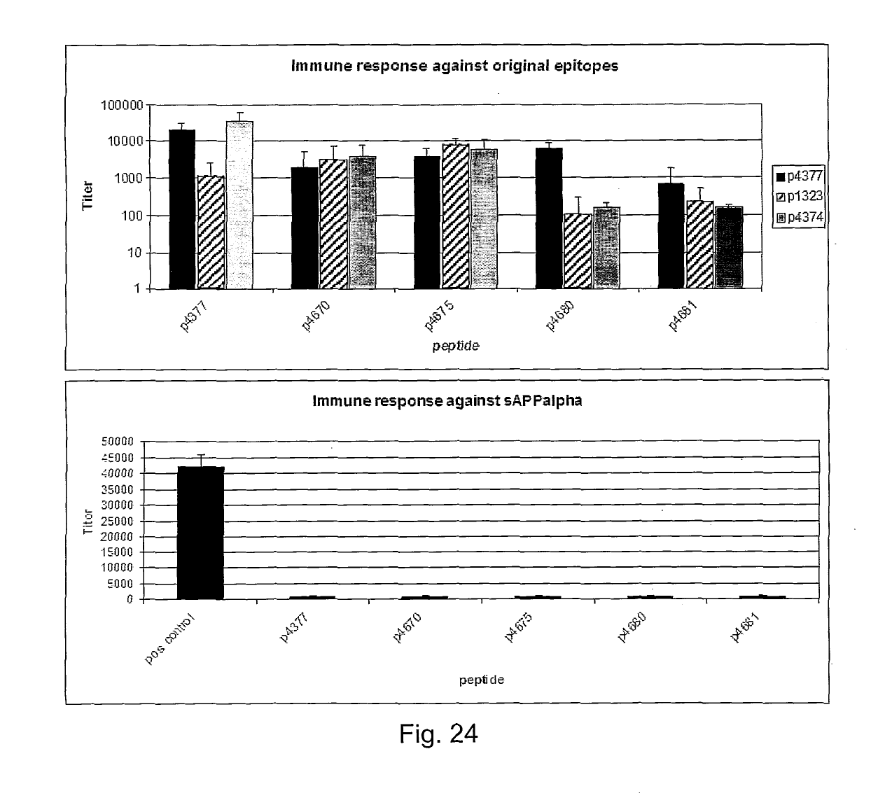

FIG. 24 shows examples for in vivo characterisation of the immune response elicited by mimotope vaccination against Amyloid Beta fragments and sAPP-alpha.

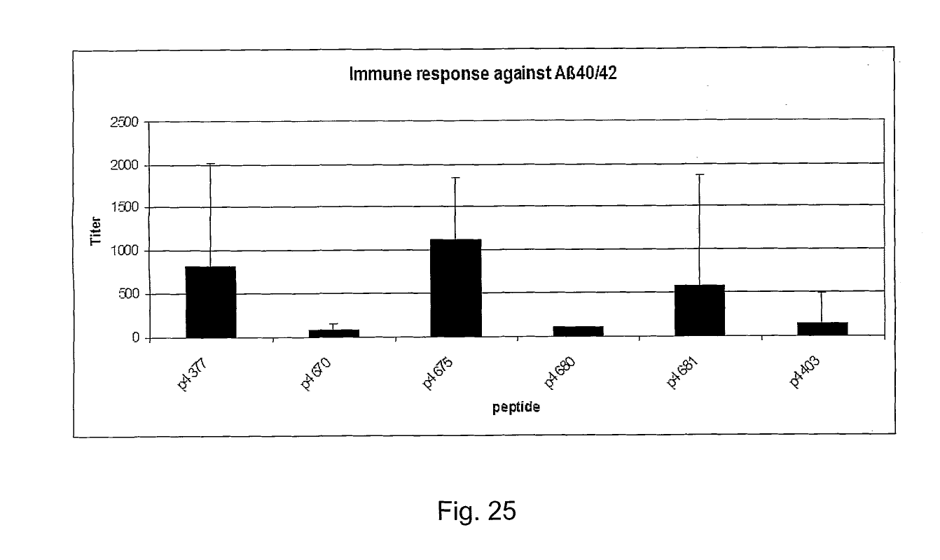

FIG. 25 shows examples for in vivo characterisation of the immune response elicited by mimotope vaccination against full length Aβ40/42.

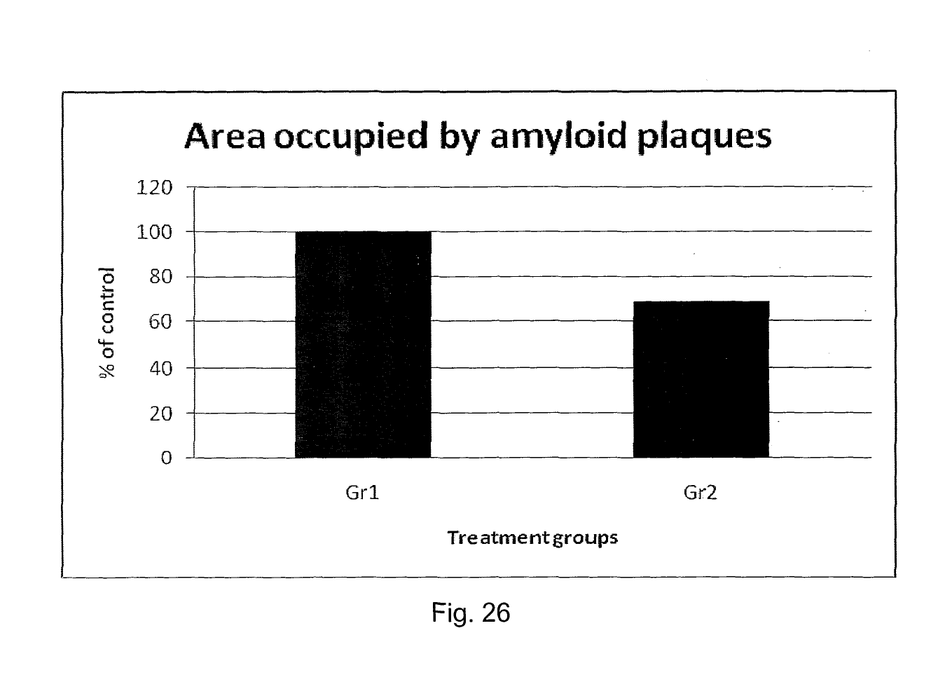

FIG. 26 shows areas occupied by amyloid plaques. Tg2576 were injected 6 times with mimotope vaccines adjuvanted with aluminium hydroxide (ALUM) by s.c. inoculation at monthly intervals. Control mice received PBS-ALUM only. Area occupied by amyloid plaques shown as percent of the control group. Gr1 . . . control group; Gr2 . . . received p4675.

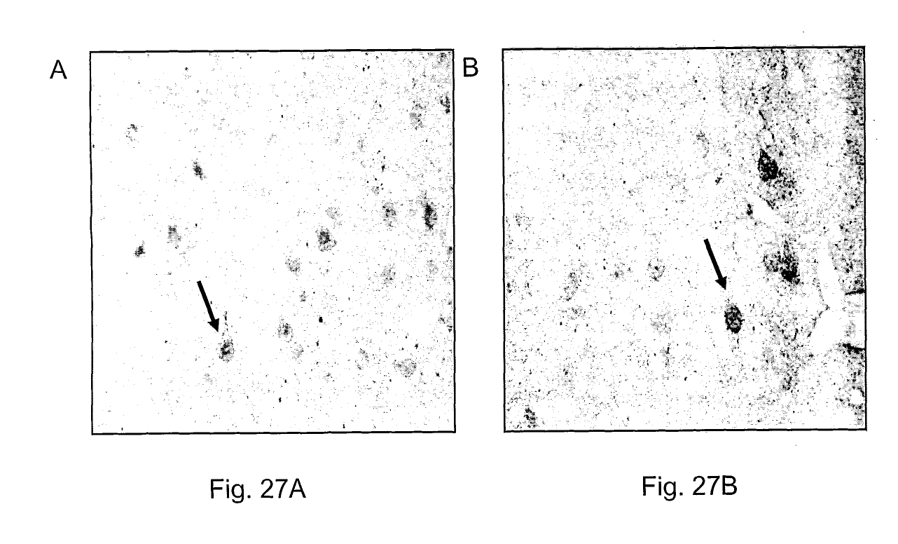

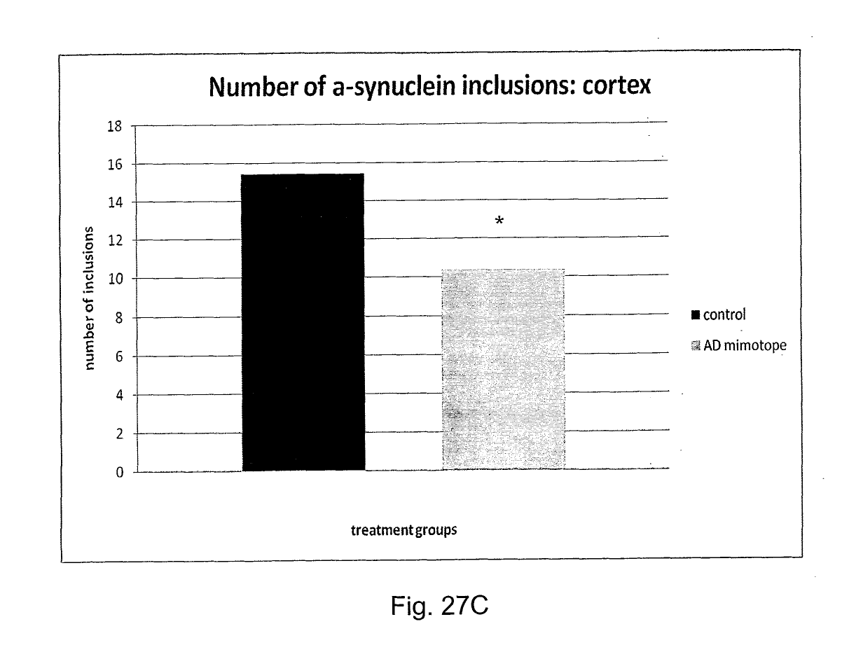

FIG. 27 shows a-synuclein positive inclusions. A . . . Control treated animal; B . . . AD mimotope treated animal; A and B display cortical sections stained for a-synuclein. Positive staining shows neuronal cells including pyramidal and non-pyramidal neurons. Arrows indicate two typical examples for inclusions in A and B. C . . . Number of inclusions in cortex and hippocampus (indicated as cortex).

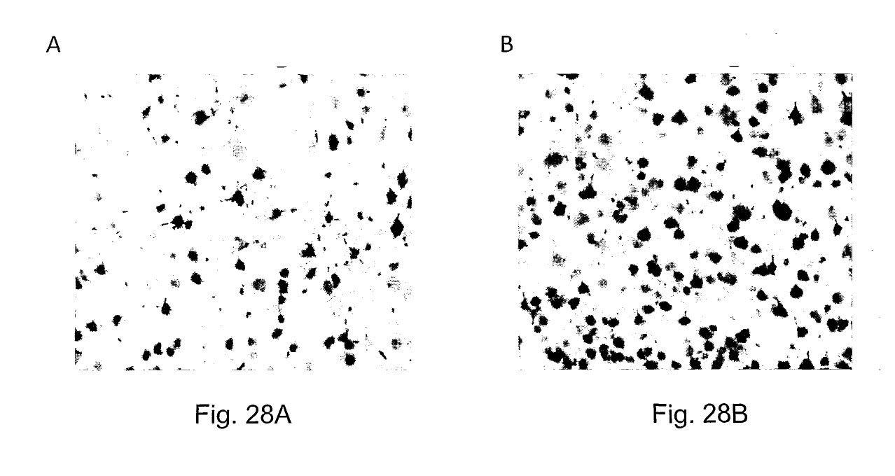

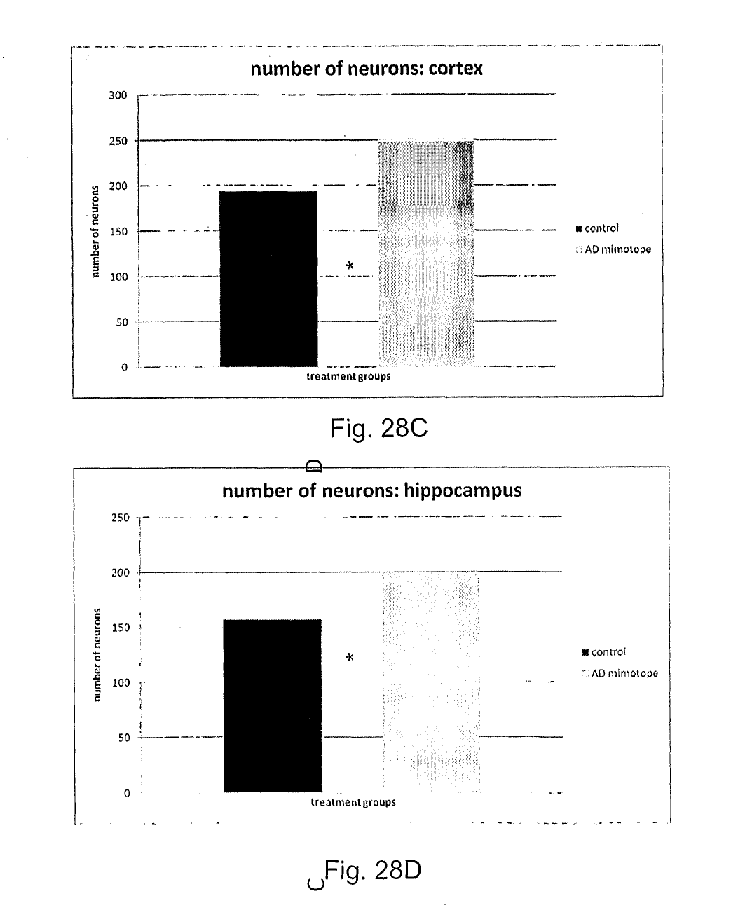

FIG. 28 shows neuronal density. Pictures display cortical sections stained for NeuN. positive staining shows neuronal cells including pyramidal and non-pyramidal neurons. A . . . indicates a control treated animal; B . . . Shows an AD mimotope treated animal respectively. C and D . . . shows the the number of NeuN positive neurons in the cortex and hippocampus.

### EXAMPLES

### Example 1

### Generation of Monoclonal Antibodies (mAb) to Detect Aβ42-Derived Peptide Species with Free N-Terminus (Free Aspartic Acid at the N-Terminus)

Mice are vaccinated with the timer peptide DAEFRH (natural N-terminal Aβ42 sequence) linked to the protein bovine serum albumin BSA (to make use of the hapten-carrier-effect), emulsified in CFA (first injection) and IFA (booster injections). DAEFRH-peptide-specific, antibody-producing hybridomas are detected by ELISA (DAEFRH-peptide-coated ELISA plates). Peptide SEVIKMEDAEFRH (natural N-terminally prolonged sequence, APP-derived, containing the Aβ42-derived sequence DAEFRH) is used as negative control peptide: hybridomas recognizing the prolonged peptide are excluded because they do not distinguish between Aβ42-derived peptides with free aspartic acid at the N-terminus and APP-derived peptide DAEFRH without free aspartic acid.

### Example 2

### Identifying Mimotopes by Inhibition Assay

3.1. Libraries

The peptide libraries employed in inhibition assays (see below) are disclosed in WO 2004/062556.

3.2. Inhibition assay

FIGS. 2 and 3 describe the results of inhibition assays performed with mimotope peptides included in and obtained from the 5 libraries (as described in WO 2004/062556). The mimotope peptides compete with the original epitope for recognition by the monoclonal antibody. Original epitope and mimotope peptides contain an additional C at the C-terminus for coupling to a protein carrier (if desired).

The following peptides are used:

| | |
|---|---|
| Peptide 1737 | DAEFRH |
| Peptide 3001 | DKELRI |
| Peptide 3002 | DWELRI |
| Peptide 3003 | YREFFI |
| Peptide 3004 | YREFRI |
| Peptide 3005 | YAEFRG |
| Peptide 3006 | EAEFRG |
| Peptide 3007 | DYEFRG |
| Peptide 3008 | ELEFRG |
| Peptide 3009 | SFEFRG |
| Peptide 3010 | DISFRG |
| Peptide 3011 | DIGWRG |

Procedure:

ELISA plates (Nunc Maxisorp) are coated with the original peptide epitope DAEFRH (C-terminally prolonged with C and coupled to bovine serum albumin BSA) at a concentration of 0.1 μg/ml peptide-BSA (100 μl/well, 12 h, 4° C.). After blocking with PBS/BSA 1% (200 μl/well, 12 h, 4° C.), the plates are washed 3× times with PBS/Tween. Then, biotinylated monoclonal antibody (1:2000, 50 μl/well) and peptides (50 μl/well) at 50, 5, 0.5, 0.05, 0.005, and 0.0005 μg/ml are added for 20 min. at 37° C. The plates are washed 3× times with PBS/Tween and are incubated with horseradish peroxidase (HRP)-labeled streptavidin (100 μl/well, 30 min, RT). The plates are washed 5× times with PBS/Tween and are incubated with ABTS+H₂O₂ (0.1% w/v, 10 to 45 min) and the reaction is stopped with citric acid followed by photometric evaluation (wavelength 405 nm).

As expected and seen in FIG. 2, peptide 1737 DAEFRH can compete with BSA-coupled, plate-bound peptide DAEFRH and thus inhibits recognition by the monoclonal antibody. Furthermore, it is shown that peptide 3003 is not able to inhibit binding of the monoclonal antibody to the original epitope. In contrast, peptides 3001, 3002, 3004, 3005, 3006, and 3007 (to a different extent) block epitope recognition. Whereas peptide 3004 is only inhibitory at a high concentration (50 μg/ml), peptides 3001, 3006, and 3007 are strongly inhibitory with an IC₅₀ of less than 0.5 μg/ml. Peptides 3002 and 3005 are “intermediate” inhibitors with an IC₅₀ of more than 0.5 μg/ml.

As expected and seen in FIG. 3, peptide 1737 DAEFRH can successfully compete with BSA-coupled, plate-bound peptide DAEFRH for monoclonal antibody recognition in an additionally performed, independent experiment. Furthermore, it is shown that peptides 3010 and 3011 are not inhibitory at the concentrations tested, whereas peptides 3008 and 3009 are (relatively) weak inhibitors with an IC₅₀ of less than 5 μg/ml.

Table 1 briefly summarizes the inhibitory capacity of mimotopes included in and obtained from libraries (as described):

TABLE 1
Inhibitory capacity of mimotopes:

| | | |
|---|---|---|
| Peptide 3001 | DKELRI | strong |
| Peptide 3002 | DWELRI | intermediate |
| Peptide 3003 | YREFFI | none |
| Peptide 3004 | YREFRI | weak |
| Peptide 3005 | YAEFRG | intermediate |
| Peptide 3006 | EAEFRG | strong |
| Peptide 3007 | DYEFRG | strong |
| Peptide 3008 | ELEFRG | weak |
| Peptide 3009 | SFEFRG | weak |
| Peptide 3010 | DISFRG | none |
| Peptide 3011 | DIGWRG | none |

### Example 3

### Inhibition Assay for Additional Mimotopes Screenend According to the Present Invention

Inhibition Assay

FIGS. 4 and 5 describe the results of inhibition assays performed with mimotope peptides included in and obtained from the 5 libraries as described in WO 2004/062556. The mimotope peptides compete with the orginal epitope for recognition by the monoclonal antibody. Original epitope and mimotope peptides contain an additional C at the C-terminus (position 7) for coupling to a protein carrier (if desired).

The following peptides are used:

| | |
|---|---|
| Peptide 1737 | DAEFRH (original epitope + C) |
| Peptide 1234 | KKELRI |
| Peptide 1235 | DRELRI |
| Peptide 1236 | DKELKI |
| Peptide 1237 | DRELKI |
| Peptide 1238 | DKELR |
| Peptide 1239 | EYEFRG |
| Peptide 1241 | DWEFRDA |
| Peptide 4002 | SWEFRT |
| Peptide 4003 | GREFRN |
| Peptide 4004 | WHWSWR |

Procedure:

ELISA plates (Nunc Maxisorp) are coated with the original peptide epitope DAEFRH (C-terminally prolonged with C and coupled to bovine serum albumin BSA) at a concentration of 0.1 μg/ml peptide-BSA (100 μl/well, 12 h, 4° C.). After blocking with PBS/BSA 1% (200 μl/well, 12 h, 4° C.), the plates are washed 3× times with PBS/Tween. Then, biotinylated monoclonal antibody (1:2000, 50 μl/well) and peptides (50 μl/well) at different concentrations are added for 20 min. at 37° C. The plates are washed 3× times with PBS/Tween and are incubated with horseradish peroxidase (HRP)-labeled streptavidin (100 μl/well, 30 min, RT). The plates are washed 5× times with PBS/Tween and are incubated with ABTS+H₂O₂ (0.1% w/v, 10 to 45 min) and the reaction is stopped with citric acid followed by photometric evaluation (wavelength 405 nm).

As expected and seen in FIG. 4, peptide 1737 DAEFRH can compete with BSA-coupled, plate-bound peptide DAEFRH and thus inhibits recognition by the monoclonal antibody. Furthermore, it is shown that peptide 4004 is not able to inhibit binding of the monoclonal antibody to the original epitope. In contrast, peptides 4002 and 4003 (to a different extent) block epitope recognition. Whereas peptide 4003 is only inhibitory at a relatively high concentration (10 μg/ml), peptide 4002 is strongly inhibitory with an IC₅₀ of less than 0.4 μg/ml.

As expected and seen in FIG. 5, peptide 1737 DAEFRH can successfully compete with BSA-coupled, plate-bound peptide DAEFRH for monoclonal antibody recognition in an additionally performed, independent experiment. Furthermore, it is shown that peptide 1234 is hardly inhibitory at the concentrations tested, whereas peptides 1235, 1236, 1237, 1238, 1239 and 1241 (to a different extent) block epitope recognition. Peptides 1235, 1238 and 1241 are strong inhibitors with an IC₅₀ of less than 0.5 μg/ml, whereas peptides 1236 and 1237 are (relatively) weak inhibitors with an IC₅₀ of more than 5 μg/ml. Peptide 1239 is an intermediate inhibitor with an IC₅₀ of more than 0.5 μg/ml.

Table 2 briefly summarizes the inhibitory capacity of mimotopes included in and obtained from libraries (as described):

TABLE 2
Inhibitory capacity of mimotopes:

| | | |
|---|---|---|
| Peptide 1234 | KKELRI | none |
| Peptide 1235 | DRELRI | strong |
| Peptide 1236 | DKELKI | weak |
| Peptide 1237 | DRELKI | weak |
| Peptide 1238 | DKELR | strong |
| Peptide 1239 | EYEFRG | intermediate |
| Peptide 1241 | DWEFRDA | strong |
| Peptide 4002 | SWEFRT | strong |
| Peptide 4003 | GREFRN | weak |
| Peptide 4004 | WHWSWR | none |

The results presented in FIGS. 4 and 5 show that in addition to various 6mer peptides (as shown here and before), 5mer peptides (namely peptide 1238 DKELR) and 7mer peptides (namely peptide 1241 DWEFRDA) may be used as epitopes in a mimotope-based Alzheimer vaccine.

### Example 4

### Inhibition Assay for Mimotopes of the Present Invention and Disclosed in WO 2006/005707

Libraries:

The mimotopes are obtained as described in WO 2006/005707.

The following peptides are used for the following assays:

| Peptide 1737 | DAEFRH | original epitope | |
|---|---|---|---|
| Peptide 4011 | DAEFRWP | 7 mer | s |
| Peptide 4012 | DNEFRSP | 7 mer | s |
| Peptide 4013 | GSEFRDY | 7 mer | m |
| Peptide 4014 | GAEFRFT | 7 mer | m |
| Peptide 4015 | SAEFRTQ | 7 mer | s |
| Peptide 4016 | SAEFRAT | 7 mer | s |
| Peptide 4017 | SWEFRNP | 7 mer | s |
| Peptide 4018 | SWEFRLY | 7 mer | s |
| Peptide 4019 | SWFRNP | 6 mer | — |
| Peptide 4020 | SWELRQA | 7 mer | s |
| Peptide 4021 | SVEFRYH | 7 mer | s |
| Peptide 4022 | SYEFRHH | 7 mer | s |
| Peptide 4023 | SQEFRTP | 7 mer | s |
| Peptide 4024 | SSEFRVS | 7 mer | s |
| Peptide 4025 | DWEFRD | timer | s |
| Peptide 4031 | DAELRY | 6 mer | s |
| Peptide 4032 | DWELRQ | 6 mer | s |
| Peptide 4033 | SLEFRF | 6 mer | s |
| Peptide 4034 | GPEFRW | 6 mer | s |
| Peptide 4035 | GKEFRT | 6 mer | s |
| Peptide 4036 | AYEFRH | 6 mer | m |
| Peptide 4037 | VPTSALA | 7 mer | — |
| Peptide 4038 | ATYAYWN | 7 mer | — |

Furthermore, the following 5mer peptides (with non natural amino acids) are used for inhibition assays:

| | | | |
|---|---|---|---|
| Peptide 4061 | DKE(tBuGly)R | 5 mer | — |
| Peptide 4062 | DKE(Nle)R | 5 mer | m |
| Peptide 4063 | DKE(Nva)R | 5 mer | m |
| Peptide 4064 | DKE((Cha)R | 5 mer | m |

(s: strong inhibition, m: moderate inhibition;
—: no inhibition)

Procedure:

ELISA plates (Nunc Maxisorp) are coated with the original peptide epitope DAEFRH (C-terminally prolonged with C and coupled to bovine serum albumin BSA) at a concentration of 0.1 μg/ml peptide-BSA (100 μl/well, 12 h, 4° C.). After blocking with PBS/BSA 1% (200 μl/well, 12 h, 4° C.), the plates are washed 3× times with PBS/Tween. Then, biotinylated monoclonal antibody (1:2000, 50 μl/well) and peptides (50 μl/well) at different concentrations are added for 20 min. at 37° C. The plates are washed 3× times with PBS/Tween and are incubated with horseradish peroxidase (HRP)-labeled streptavidin (100 μl/well, 30 min, RT). The plates are washed 5× times with PBS/Tween and are incubated with ABTS+H₂O₂ (0.1% w/v, 10 to 45 min) and the reaction is stopped with citric acid followed by photometric evaluation (wavelength 405 nm).

As expected and seen in FIG. 6 (showing peptides 4011-4018), peptide 1737 DAEFRH can compete with BSA-coupled, plate-bound peptide DAEFRH and thus inhibits recognition by the monoclonal antibody. Furthermore, it is shown that peptides 4012 DNEFRSP, 4013 GSEFRDY, and 4014 GAEFRFT are able to moderately inhibit binding of the monoclonal antibody to the original epitope. In contrast, peptides 4011 DAEFRWP, 4015 SAEFRTQ, 4016 SAEFRAT, 4017 SWEFRNP, and 4018 SWEFRLY (to a different extent) strongly block epitope recognition.

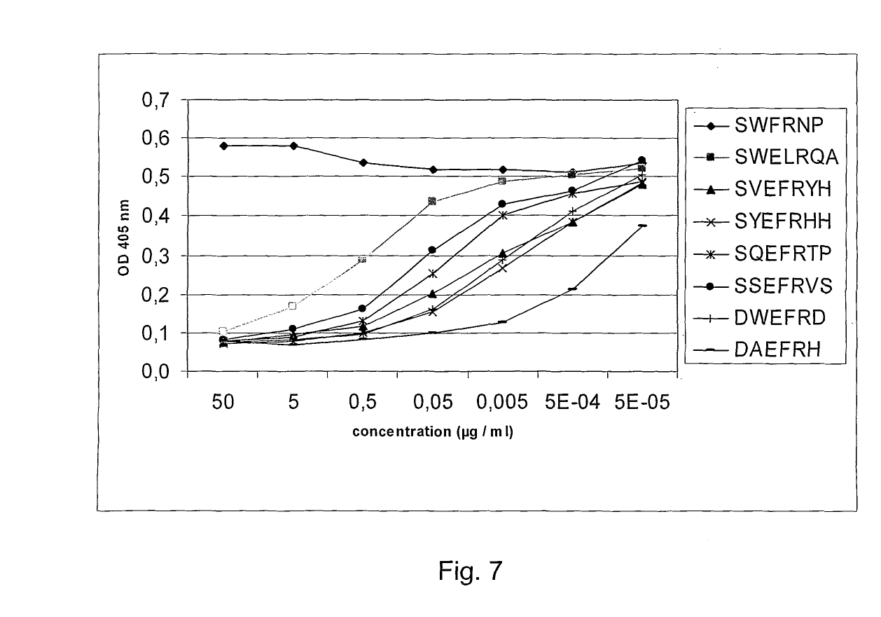

As expected and presented in FIG. 7 (showing peptides 4019-4025), peptide 1737 DAEFRH can successfully compete with BSA-coupled, plate-bound peptide DAEFRH for monoclonal antibody recognition in an additionally performed, independent experiment. Furthermore, it is shown that peptide 4019 SWFRNP is not inhibitory at the concentrations tested, whereas peptides 4020 SWELRQA, 4021 SVEFRYH, 4022 SYEFRHH, 4023 SQEFRTP, 4024 SSERFVS and 4025 DWEFRD (to a different extent) block epitope recognition. Peptides 4021, 4022, 4023, 4024 and 4025 are strong inhibitors with an IC50 of less than 0.5 μg/ml, whereas pepₜide 4020 is an intermediate inhibitor with an IC50 of more than 0.5 μg/ml.

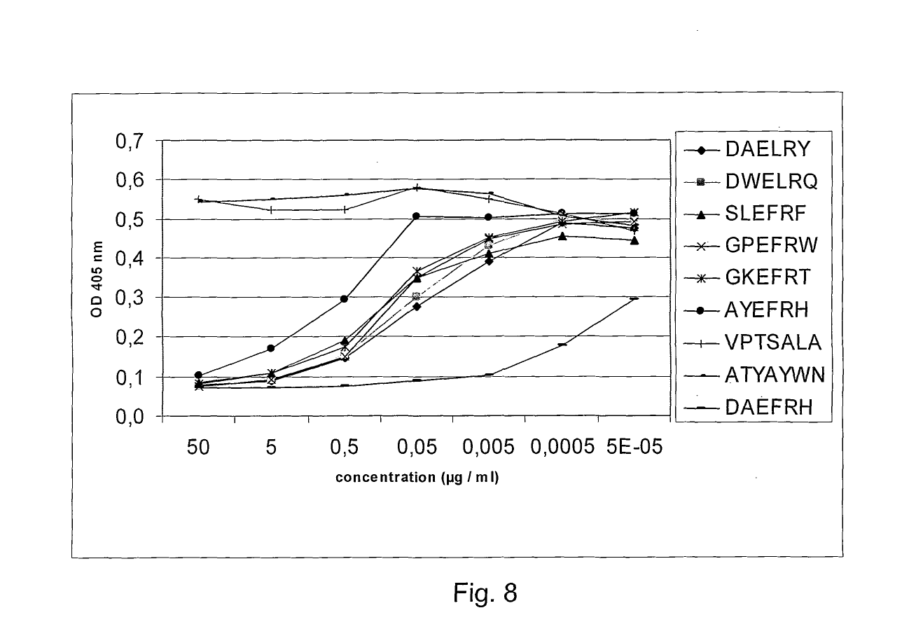

As expected and seen in FIG. 8 (peptides 4031-4038), peptide 1737 DAEFRH can successfully compete with BSA-coupled, plate-bound peptide DAEFRH for monoclonal antibody recognition in a 3rd independent experiment. Furthermore, it is shown that peptides 4037 VPTSALA and 4038 ATYAYWN are not inhibitory at the concentrations tested, whereas peptides 4031 DAELRY, 4032 DWELRQ, 4033 SLEFRF, 4034 GPEFRW, 4035 GKEFRT and 4036 AYEFRH (to a different extent) block epitope recognition. Peptides 4031, 4032, 4033, 4034 and 4035 are relatively strong inhibitors with an IC50 of less than 0.5 μg/ml, whereas peptide 4036 is a (relatively) weak inhibitor with an IC50 of more than 0.5 μg/ml.

In the following Table further examples of the immune response elicited by using AD mimotopes are described. All peptides listed in table 1 mount specific immune reactions against full length Aβ and/or fragments thereof.

| Internal Peptide number | Detection of Aβ |
|---|---|
| p1122 | + |
| p1123 | + |
| p1125 | + |
| p1238 | + |
| p1239 | + |
| p1252 | + |
| p1283 | + |
| p3005 | + |
| p3006 | + |
| p3007 | + |
| p3008 | + |
| p4003 | + |
| p4020 | + |
| p4023 | + |
| p4033 | + |
| p4034 | + |
| p4035 | + |

### Example 5

### Inhibition Assay with Defined 5mer Peptides: Non-Natural Amino Acids

It has been shown previously that the 5mer peptide 1238 DKELR may be used as epitope in a mimotope-based Alzheimer vaccine (see PCT/EPO4/00162). In the following, amino acids of the original 5mer epitope are replaced by non-natural amino acids: L is replaced by the non-natural amino acids tBuGly, Nle, Nva, or Cha.

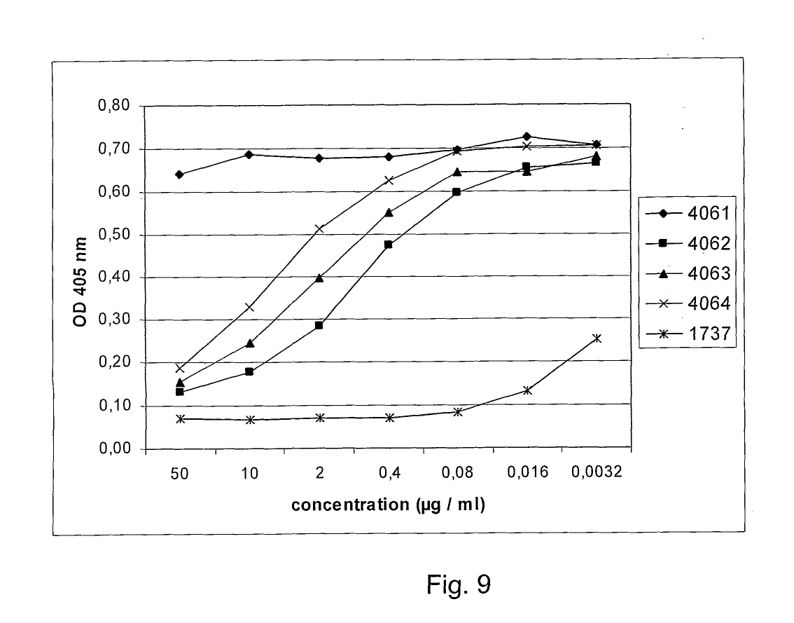

As expected and presented in FIG. 9 (peptides 4061-4064 DKELR.variants), peptide 1737 DAEFRH can successfully. compete with BSA-coupled, plate-bound peptide DAEFRH for monoclonal antibody recognition in a 4th independent experiment. Furthermore, it is shown that peptide 4061 DKE(tBuGly)R is not inhibitory at the concentrations tested. Interestingly, peptides 4062 DKE(Nle)R, 4063 DKE((Nva)R, and 4064 DKE(Cha)R (to a different extent) block epitope recognition. Peptides 4062, 4063, and 4064 are relatively weak inhibitors with an IC50 of more than 0.5 μg/ml.

### Example 6

### Generation of Monoclonal Antibodies to Specifically Detect β-Amyloid and N-Terminally Truncated and/or Post-Translationally Modified β-Amyloid Fragments

Methods

The antibodies used for the mimotope identification according to the following examples detect amino acid sequences derived from human Aβ but do not bind to full length human APP. The sequences detected include EFRHDS (=original epitope aa3-8 of Aβ), p(E)FRHDS(=original epitope of the modified aa3-8 of Aβ), EVHHQK(=original epitope aa11-16 of Aβ). The antibody may be a monoclonal or polyclonal antibody preparation or any antibody part or derivative thereof, the only prerequisite is that the antibody molecule specifically recognises at least one of the epitopes mentioned above (derived from human Aβ), but does not bind to full length human APP.

The mimotopes are identified and further characterised with such monoclonal antibodies and peptide libraries.

### Example 6a

### Generation of Monoclonal Antibody MV-001

A monoclonal antibody derived from the fusion of experiment Alz-5 was generated: In experiment Alz-5 C57/516 mice were immunized repeatedly with original AS epitope DAEFRHDSGYC coupled to KLH (Keyhole Limpet Hemocyanin) and. Alum (Aluminium Hydroxide) as adjuvant. p4371-peptide-specific, antibody-producing hybridomas were detected by ELISA (p1253- and p4371-peptide-coated ELISA plates). Human Aβ840/42 (recombinant protein) was used as positive control peptide: hybridomas recognizing the recombinant protein immobilised on ELISA plates were included because they are binding both peptide and full length AS specifically. P1454 (Human Aβ 33-40) was used as negative control peptide. Furthermore hybridomas were tested against p4373. Only hybridomas with no or limited p4373 binding were used for further antibody development.

The Hybridoma clone (MV-001 (internal name 824; IgG1) was purified and analysed for specific detection of p1253, p4371, p4373, p1454 and Aβ respectively. MV-001 recognized the injected epitope (p1253) as well as the specific epitope (p4371) and full length Aβ protein (recombinant protein; obtained from Bachem AG, Bubendorf, Switzerland) in ELISA. It however did not detect p1454 in ELISA. Furthermore, the MV-001 antibodies basically failed to detect the peptide p4373 encoding the pyroglutamate version of Aβ3-10 (30 times lower titer than the original epitopes).

### Example 6b

### Generation of Monoclonal Antibody MV-003

A monoclonal antibody derived from the fusion of experiment Alz-16 was generated: In experiment Alz-16 BalbC mice were immunized repeatedly with the epitope p(E)FRHDSC (p4373) coupled to KLH (Keyhole Limpet Hemocyanin) and Alum (Aluiminium Hydroxide) as adjuvant. p4373-peptide-specific, antibody-producing hybridomas were detected by ELISA (p4373-peptide-coated ELISA plates). p1253, p1454 and Aβ40/42 were used as negative control peptides. Furthermore, hybridomas were tested against p4371. Only hybridomas with no or limited p4371 binding were used for further antibody development in order to guarantee for pyroglutamate-specificity.

The Hybridoma clone (MV-003 (internal name D129; IgG1) was purified and analysed for specific detection of p1253, p4371, p4373, p1454 and Aβ respectively. MV-003 recognized the injected epitope (p4373) but failed to detect p1454, p1253 or full length Aβ protein (recombinant protein; obtained from Bachem AG, Bubendorf, Switzerland) in ELISA. Furthermore, the MV-003 antibodies failed to detect the peptide p4371 encoding the normal version of Aβ3-10 (15 times lower titer than the original epitope).

### Example 6c

### Generation of Monoclonal Antibody MV-004

A monoclonal antibody derived from the fusion of experiment Alz-15 was generated: In experiment Alz-15 BalbC mice were immunized repeatedly with the epitope EVHHQKC (p4372) coupled to KLH (Keyhole Limpet Hemocyanin) and Alum (Aluiminium Hydroxide) as adjuvant. p4372-peptide-specific, antibody-producing hybridomas were detected by ELISA (p4372-peptide-coated ELISA plates). P4376, p4378, p1454 and Aβ40/42 were used as negative control peptides. Only hybridomas with no or limited p4376 and p4378 binding were used for further antibody development in order to guarantee for specificity against the free N-Terminus at position aa11.

The Hybridoma clone (MV-004 (internal name B204; IgG1) was purified and analysed for specific detection of p4372, p4376, p4378, p1454 and Aβ respectively. MV-004 recognized the injected epitope (p4372) but failed to detect p1454, p4376 and p4378 as well as full length Aβ protein (recombinant protein; obtained from Bachem AG, Bubendorf, Switzerland) in ELISA. The failure to detect p4376, p4378 demonstrates specificity for the free N-terminus at position aa11 in truncated Aβ.

### Example 6d

### Generation of Monoclonal Antibodies to Specificcally Detect β-Amyloid and N-Terminally Truncated and/or Post-Translationally Modified β-Amyloid Fragments—Monoclonal Antibody MV-002

Methods

The antibodies used for the mimotope identification according to the present invention detect amino acid sequences derived from human Aβ but do not bind to full length human APP. The sequences detected include EVHHQKLVFFAED (=original epitope aa11-24 of Aβ) and p(E)VHHQKLVF (p4374=original epitope aa11-19 of Aβ with a pyroglutamate modification at the N-Terminus). The antibody may be a monoclonal or polyclonal antibody preparation or any antibody part or derivative thereof, the only prerequisite is that the antibody molecule specifically recognises at least one of the epitopes mentioned above (derived from human Aβ), but does not bind to full length human APP.

The mimotopes are identified and further characterised with such monoclonal antibodies and peptide libraries.

A monoclonal antibody derived from the fusion of experiment Alz-9 was generated: C57/B16 mice were immunized repeatedly with original Aβ epitope HQKLVFC coupled to KLH (Keyhole Limpet Hemocyanin) and Alum (Aluiminium Hydroxide) as adjuvant. p4377 pep-tide-specific, antibody-producing hybridomas were detected by ELISA (p4377-peptide-coated ELISA plates). Human Aβ40/42 (recombinant protein) was used as positive control peptide: hybridomas recognizing the recombinant protein immobilised on ELISA plates were included because they were binding both peptide and full length Aβ specifically. p1454 (Human Aβ 33-40) was used as negative control peptide. Furthermore hybridomas were tested against p4374, p1323 and sAPP-alpha. Only hybridomas with good p4374, and p1323 binding and a lack of sAPP-alpha binding were used for further antibody development.

The Hybridoma clone MV-002 (internal name A115; IgG2b) was purified and analysed for specific detection of p1323, p4374, p4377, p1454, Aβ and sAPP-alpha respectively. MV-002 recognized the epitopes p1323 as well as p4377 and full length Aβ protein (recombinant protein; obtained from Bachem AG, Bubendorf, Switzerland) in ELISA. It however did not detect p1454 in ELISA. Furthermore, the MV-002 antibodies failed to detect sAPP-alpha but bound specifically to the peptide p4374 encoding the pyroglutamate version of Aβ11-19.

### Example 7

### Phage Display, In Vitro Binding and Inhibition ELISA

Phage Display libraries used in this example were: Ph.D. 7: New England BioLabs E8102L (linear 7mer library). Phage Display was done according to manufacturer's protocol (www.neb.com).

After 2 or 3 subsequent rounds of panning, single phage clones were picked and phage supernatants were subjected to ELISA on plates coated with the antibody that was used for the panning procedure. Phage clones that were positive in this ELISA (strong signal for the target, but no signal for unspecific control) were sequenced. From DNA sequences, peptide sequences were deduced. These peptides were synthesized and characterised in binding and inhibition ELISA. Additionally, some novel mimotopes were created by combining sequence information from mimotopes identified in the screen to support the identification of a consensus sequence for a mimotope vaccination.

1. In Vitro Binding Assay (ELISA)

Peptides derived from Phage Display as well as variants thereof were coupled to BSA and bound to ELISA plates (1 μM; as indicated in the respective figures) and subsequently incubated with the monoclonal antibody that was used for the screening procedure to analyse binding capacity of identified peptides.

2. In Vitro Inhibition Assay (ELISA)

Different amounts of peptides (concentrations ranging from 10 μg to 0.08 μg; serial dilutions; for MV-002: concentrations ranging from 5 μg to 0.03 μg; serial dilutions), derived from Phage Display were incubated with the monoclonal antibody that was used for the screening procedure. Peptides diminishing subsequent binding of the antibody to the original epitope coated on ELISA plates were considered as inhibiting in this assay.

### Example 8

### In Vivo Testing of Mimotopes: Analysis of Immunogenicity and Crossreactivity

1. In Vivo Testing of Mimotopes

Inhibiting as well as non-inhibiting peptides were coupled to KLH and injected into mice (wildtype C57/B16 mice; subcutaneous injection into the flank) together with an appropriate adjuvant (aluminium hydroxide). Animals were vaccinated 3-6 times in biweekly intervals and sera were taken biweekly as well. Titers to injected peptides, as well as to an irrelevant peptide were determined with every serum. Furthermore, titers against the recombinant human Aβ protein, and against original peptides were determined respectively. In general sera were analysed by reaction against peptides coupled to Bovine Serum Albumin (BSA) and recombinant full length proteins which were immobilised on ELISA plates. Titers were determined using anti mouse IgG specific antibodies. For detailed results see FIGS. 15, 16 and 17 respectively and FIGS. 23, 24 and 25 respectively.

2. Results for MV-001, MV-003 and MV-004

2.1. Identification of Specific Monoclonal Antibodies (mAB) Directed Against N-Terminally Truncated and Modified Forms of Aβ:

FIG. 10 depicts the characterisation of the monoclonal antibody MV-001 (internal name 824; IgG1) derived from experiment Alz-5 demonstrating specificity for full length Aβ and Aβ truncated at position E3.

FIG. 11 depicts the characterisation of the monoclonal antibody MV-003 (internal name D129; IgG1) derived from experiment Alz-16 demonstrating specificity for Aβ truncated and posttranslationally modified at position p(E)3.

FIG. 12 depicts the characterisation of the monoclonal antibody MV-004 (internal name B204; IgG1) derived from experiment Alz-15 demonstrating specificity for Aβ truncated at position E11.

2.2. Screening with Specific mABs Directed Against N-Terminally Truncated and Modified Forms of Aβ:

2.2.1. Phage Display Library Ph.D. 7

2.2.1.1. Screening with Monoclonal Antibody Directed Against p4373

8 Sequences were identified by screening PhD 7 phage display libraries in this screen: Table 1A summarises the peptides identified and their binding capacity as compared to the original epitope.

2.2.1.2. Screening with Monoclonal Antibody Directed Against p4372

9 Sequences were identified by screening PhD 7 phage display libraries in this screen: Table 1B summarises the peptides identified and their binding capacity as compared to the original epitope.

2.2.1.3. Screening with Monoclonal Antibody Directed Against p4371

71 Sequences were identified by screening PhD 7 and PhD12 phage display libraries in this screen: Table 1C summarises the peptides identified and their binding capacity as compared to the original epitope.

TABLE 1A
mimotopes binding to the parental antibody MV-003

| Internal Peptide | | Binding |
|---|---|---|
| number | Sequence | Capacity |
| p4395 | IRWDTPC | 2 |
| p4396 | VRWDVYPC | 1 |
| p4397 | IRYDAPLC | 1 |
| p4399 | IRYDMAGC | 1 |
| p4728 | IRWDTSLC | 3 |
| p4756 | IRWDQPC | 3 |
| p4792 | IRWDGC | 1 |
| p4793 | IRWDGGC | 2 |

Legend to Table 1A: the binding capacity is coded
by the following binding code: 1:X describes
the dilution factor of the parental AB.

| | | OD halfmax |
|---|---|---|
| binding code | | 1:X |
| 0 | no binding | :0 |
| 1 | weak binding | :<16000 |
| 2 | medium binding | :16-60000 |
| 3 | strong binding | :>60000 |

TABLE 1B
mimotopes binding to the parental antibody MV-004

| Internal Peptide | | Binding |
|---|---|---|
| number | Sequence | Capacity |
| p4417 | EVWHRHQC | 2 |
| p4418 | ERWHEKHC | 3 |
| p4419 | EVWHRLQC | 3 |
| p4420 | ELWHRYPC | 2 |
| p4665 | ELWHRAFC | 2 |
| p4786 | ELWHRAC | 1 |
| p4788 | EVWHRHC | 1 |
| p4789 | EVWHRHC | 1 |
| p4790 | ERWHEKC | 1 |

Legend to Table 1B: the binding capacity is
coded by the following binding code: 1:X
describes the dilution factor of the parental AB.

| binding code | | OD halfmax 1:X |
|---|---|---|
| 0 | no binding | :0 |
| 1 | weak binding | :<24000 |
| 2 | medium binding | :24-96000 |
| 3 | strong binding | :>96000 |

TABLE 1C
mimotopes binding to the parental antibody MV-001

| Internal Peptide | | Binding |
|---|---|---|
| number | Sequence | Capacity |
| p4380 | QDFRHYC | 2 |
| p4381 | SEFKHGC | 3 |
| p4382 | TSFRHGC | 2 |
| p4383 | TSVFRHC | 3 |
| p4384 | TPFRHTC | 2 |
| p4385 | SQFRHYC | 2 |
| p4386 | LMFRHNC | 3 |
| p4387 | SAFRHHC | 2 |
| p4388 | LPFRHGC | 2 |
| p4389 | SHFRHGC | 2 |
| p4390 | ILFRHGC | 3 |
| p4391 | QFKHDLC | 2 |
| p4392 | NWFPHPC | 1 |
| p4393 | EEFKYSC | 2 |
| p4701 | NELRHSTC | 3 |
| p4702 | GEMRHQPC | 3 |
| p4703 | DTYFPRSC | 2 |
| p4704 | VELRHSRC | 2 |
| p4705 | YSMRHDAC | 2 |
| p4706 | AANYFPRC | 2 |
| p4707 | SPNQFRHC | 3 |
| p4708 | SSSFFPRC | 2 |
| p4709 | EDWFFWHC | 1 |
| p4710 | SAGSFRHC | 3 |
| p4711 | QVMRHHAC | 2 |
| p4712 | SEFSHSSC | 3 |
| p4713 | QPNLFYHC | 1 |
| p4714 | ELFKHHLC | 3 |
| p4715 | TLHEFRHC | 3 |
| p4716 | ATFRHSPC | 2 |
| p4717 | APMYFPHC | 2 |
| p4718 | TYFSHSLC | 2 |
| p4719 | HEPLFSHC | 1 |
| p4721 | SLMRHSSC | 2 |
| p4722 | EFLRHTLC | 3 |
| p4723 | ATPLFRHC | 3 |
| p4724 | QELKRYYC | 1 |
| p4725 | THTDFRHC | 3 |
| p4726 | LHIPFRHC | 3 |
| p4727 | NELFKHFC | 2 |
| p4729 | SQYFPRPC | 2 |
| p4730 | DEHPFRHC | 3 |
| p4731 | MLPFRHGC | 2 |
| p4732 | SAMRHSLC | 2 |
| p4733 | TPLMFWHC | 1 |
| p4734 | LQFKHSTC | 2 |
| p4735 | ATFRHSTC | 2 |
| p4736 | TGLMFKHC | 2 |
| p4737 | AEFSHWHC | 2 |
| p4738 | QSEFKHWC | 3 |
| p4739 | AEFMHSVC | 2 |
| p4740 | ADHDFRHC | 3 |
| p4741 | DGLLFKHC | 3 |
| p4742 | IGFRHDSC | 2 |
| p4743 | SNSEFRRC | 3 |
| p4744 | SELRHSTC | 3 |
| p4745 | THMEFRRC | 3 |
| p4746 | EELRHSVC | 3 |
| p4747 | QLFKHSPC | 3 |
| p4748 | YEFRHAQC | 3 |
| p4749 | SNFRHSVC | 3 |
| p4750 | APIQFRHC | 3 |
| P4751 | AYFPHTSC | 2 |
| p4752 | NSSELRHC | 3 |
| p4753 | TEFRHKAC | 3 |
| p4754 | TSTEMWHC | 1 |
| p4755 | SQSYFKHC | 3 |
| p4800 | CSEFKH | 3 |
| p4801 | SEFKHC | 3 |
| p4802 | CHEFRH | 3 |
| p4803 | HEFRHC | 3 |

Legend to Table 10: the binding capacity is coded
by the following binding code: 1:X describes the
dilution factor of the parental AB

| binding code | | OD halfmax 1:X |
|---|---|---|
| 0 | no binding | :0 |
| 1 | weak binding | :<4000 |
| 2 | medium binding | :4000-20000 |
| 3 | strong binding | :>20000 |

2.3. In Vitro Characterisation of Mimotopes Identified in Screening Phage Display Libraries with Monoclonal Antibodies Directed against N-Terminally Truncated and Modified Forms of Aβ:

FIGS. 13 and 14 show representative examples for binding and inhibition assays used to characterise mimotopes in vitro. Data obtained are summarised in Tables 1 and 2 respectively.

MV-003 Mimotopes: From the 8 sequences presented 6 sequences inhibit binding of the p(E)3-7Aβ specific monoclonal antibody in in vitro competition experiments: Additional 2 sequences were identified that do not inhibit binding of monoclonal antibody in in vitro competition experiments but still retain binding capacity to the parental antibody (Table 2A).

MV-004 Mimotopes: All the 9 sequences presented inhibit binding of the monoclonal antibody specifically binding the free N-terminus of Aβ truncated at position E11 in in vitro competition experiments: (Table 2B).

MV-001 Mimotopes: From the 71 sequences presented 27 sequences inhibit binding of the monoclonal antibody specifically directed against Aβ truncated at position E3 in in vitro competition experiments: Additional 44 sequences were identified that do not inhibit binding of monoclonal antibody in in vitro competition experiments but still retain binding capacity to the parental antibody (Table 2C).

Table 2: mimotopes identified in this invention giving positive results in inhibiting assays

TABLE 2A
MV-003 Mimotopes

| Internal | | |
|---|---|---|
| Peptide | | Inhibition |
| number | Sequence | Capacity |
| p4395 | IRWDTPC | 1 |
| p4397 | IRYDAPLC | 1 |
| p4728 | IRWDTSLC | 2 |
| p4756 | IRWDQPC | 1 |
| p4792 | IRWDGC | 1 |
| p4793 | IRWDGGC | 1 |

Legend to Table 2A: the inhibition capacity is
coded by the following code: Weak inhibition
means more peptide is required to lower AB
binding than with the original epitope; strong
inhibition means similar peptide amounts are
required for mimotope and original epitope for
lowering AB binding. Mimotopes are compared to
the original peptide as standard. OD at 10 μg
peptide used in the assay is used to calculate
the competition capacity compared to original
peptide.

| competition | |
|---|---|
| code | |
| 0 | no inhibition (OD of 10 μg peptide |
| | above 12 times of original peptide) |
| 1 | Weaker than original epitope (OD |
| | of 10 μg peptide below 12 times |
| | of original peptide) |
| 2 | strong inhibition (as original |
| | epitope; OD of 10 μg peptide below |
| | 5 times of original peptide) |

TABLE 2B
MV-004 Mimotopes

| Internal Peptide | | Inhibition |
|---|---|---|
| number | Sequence | Capacity |
| p4417 | EVWHRHQC | 1 |
| p4418 | ERWHEKHC | 2 |
| p4419 | EVWHRLQC | 2 |
| p4420 | ELWHRYPC | 1 |
| p4665 | ELWHRAFC | 2 |
| p4786 | ELWHRAC | 1 |
| p4788 | EVWHRGC | 1 |
| p4789 | EVWHRHC | 1 |
| p4790 | ERWHEKC | 2 |

Legend to Table 2B: the inhibition capacity is
coded by the following code: Weak inhibition
means more peptide is required to lower AB
binding than with the original epitope;
strong inhibition means similar peptide amounts
are required for mimotope and original
epitope for lowering AB binding. Mimotopes
are compared to the original peptide as
standard. OD at 10 μg peptide used in the
assay is used to calculate the competition
capacity compared to original peptide.

| competition | |
|---|---|
| code | |
| 0 | no inhibition (OD of 10 μg peptide |
| | above 5 times of original peptide) |
| 1 | Weaker than original epitope (OD of |
| | 10 μg peptide below 5 times of |
| | original peptide) |
| 2 | strong inhibition (as original epitope; |
| | OD of 10 μg peptide below 2 times of |
| | original peptide) |

TABLE 2C
MV-001 Mimotopes

| Internal Peptide | | Inhibition |
|---|---|---|
| number | Sequence | Capacity |
| p4380 | QDFRHYC | 1 |
| p4381 | SEFKHGC | 1 |
| p4382 | TSFRHGC | 1 |
| p4383 | TSVFRHC | 1 |
| p4384 | TPFRHTC | 1 |
| p4385 | SQFRHYC | 1 |
| p4386 | LMFRHNC | 1 |
| p4387 | SAFRHHC | 1 |
| p4388 | LPFRHGC | 1 |
| p4389 | SHFRHGC | 1 |
| p4390 | ILFRHGC | 1 |
| p4391 | QFKHDLC | 1 |
| p4392 | NWFPHPC | 1 |
| p4393 | EEFKYSC | 1 |
| p4707 | SPNQFRHC | 1 |
| p4715 | TLHEFRHC | 2 |
| p4725 | THTDFRHC | 1 |
| p4730 | DEHPFRHC | 1 |
| p4738 | QSEFKHWC | 1 |
| p4740 | ADHDFRHC | 1 |
| p4741 | DGLLFKHC | 1 |
| p4746 | EELRHSVC | 1 |
| p4753 | TEFRHKAC | 2 |
| p4800 | CSEFKH | 2 |
| p4801 | SEFKHC | 1 |
| p4802 | CHEFRH | 2 |
| p4803 | HEFRHC | 2 |

```
Legend to Table 2C: the inhibition capacity is
coded by the following code: Weak inhibition
means more peptide is required to lower AB
binding than with the original epitope; strong
inhibition means similar peptide amounts are
required for mimotope and original epitope for
lowering AB binding. Mimotopes are compared to
the original peptide as standard. OD at 10 μg
peptide used in the assay is used to calculate
  the competition capacity compared
  to original peptide.
```

| competition | |
|---|---|
| code | |
| 0 | no inhibition (OD of 10 μg peptide |
| | above 3 times of original peptide) |
| 1 | Weaker than original epitope (OD |
| | of 10 μg peptide below 3 times of |
| | original peptide) |
| 2 | strong inhibition (as original |
| | epitope; OD of 10 μg peptide below |
| | 2 times of original peptide) |

TABLE 3
Non-mimotope peptides

| Internal | |
|---|---|
| Peptide | |
| number | Sequence |
| p1253 | DAEFRHDSGYC |
| p4371 | EFRHDS-C |
| p4372 | EVHHQK-C |
| p4373 | p(E)FRHDS-C |
| p4374 | p(E)VHHQKLVFC |
| p4376 | GYEVHHQKC |
| p4377 | EVHHQKLVFC |
| p4378 | C-EVHHQKLVFF |
| p1454 | CGLMVGGVV |
| Aβ1-40 | DAEFRHDSGYEVHHQKLVFFAEDVGSNKGAIIGLMVGGVV |
| Aβ1-42 | DAEFRHDSGYEVHHQKLVFFAEDVGSNKGAIIGLMVGGVVIA |
| sAPPalpha | alpha-Secretase induced cleavage product |
| | derived from human APP (gi: 112927) |

2.4. In Vivo Characterisation of Mimotopes Identified in Screening Phage Display Libraries with a Monoclonal Antibody Directed against against N-Terminally Truncated and Modified Forms of Aβ:

Female C57/b16 mice, 5-6 mice per group, were subcutaneously immunized with 30 μg peptide coupled to KLH. Control groups were administered original epitope-KLH conjugates respectively. As adjuvant alum was used (always 1 mg per mouse). The peptides administered were all able to bind to monoclonal antibodies specifically although some of the peptides did not inhibit the binding of the original epitope to its parental antibody in vitro (in an in vitro inhibition assay). The in vitro ELISA assay to determine the antibody titer was performed with sera of single mice after each vaccination in a two week interval (see FIGS. 15 and 16 respectively). The wells of the ELISA plate were coated with mimotope-BSA conjugate and an irrelevant peptide-BSA conjugate (negative control). The positive control was performed by reaction of the parental antibody with the respective mimotope-BSA conjugate. The detection was performed with anti-mouse IgG. Additionally, recombinant proteins were immobilised on ELISA plates and sera reacted accordingly. FIGS. 15 to 17 show representative examples for assays used to characterise mimotopes in vivo.

FIG. 15 shows examples for in vivo characterisations of the immune response elicited by mimotope vaccination by analysing the immune response against injected peptide and an irrelevant peptide, containing an unrelated sequence. In all three examples shown, the original epitopes and the mimotopes, elicit immune responses against the injected peptides but fail to induce a relevant immune response against an unrelated sequence (p1454).

As example for MV-003-mimotopes, original epitope p4373 and the mimotopes p4395, p4396, p4397, and p4399 are depicted in FIG. 15A. All vaccines are mounting similar immune responses against their respective mimotopes. Neither original epitope p4373-vaccine treated nor the animals treated with mimotope p4395, p4396, p4397 or p4399- vaccines mount relevant titers against irrelevant peptide p1454 (11×-25× less than injected peptides).

As example for MV-004-mimotopes original epitope p4372 and the mimotopes p4417, p4418, p4419, and p4420 are depicted in FIG. 15B. All vaccines are mounting similar immune responses against their respective mimotopes. Neither original epitope p4372-vaccine treated nor the animals treated with mimotope p4417, p4418, p4419, and p4420-vaccines mount relevant titers against irrelevant peptide p1454 (20-80× less than injected peptides).

As example for MV-001-mimotopes original epitope p4371 and the mimotopes p4381, p4382, and p4390 are depicted in FIG. 15C. All vaccines are mounting similar immune responses against their respective mimotopes. Neither original epitope p4371-vaccine treated nor the animals treated with mimotope p4381, p4382, and p4390—vaccines mount relevant titers against irrelevant peptide p1454 (>10× less than injected peptides).

FIG. 16 shows examples for in vivo characterisations of the immune response elicited by mimotope vaccination against the respective original epitope of the parental antibody as well as against peptides derived of other forms of truncated species of Aβ.

As example for MV-003-mimotopes, original epitope p4373 and the mimotopes p4395, p4396, p4397, and p4399 are depicted in FIG. 16A. ¾ Mimotope vaccines indicated mount detectable immune responses against the original epitope p4373. A similar phenomenon can he detected analysing cross reactivity against the non-modified form as displayed by p4371. The original epitope p4373-vaccine and 2/4 Mimotope vaccines mount relevant titers against p4371. Surprisingly, the mimotopes selected by MV-003, which is specifically binding to p4373 are also inducing a immune reaction cross reacting with the unmodified form of the original epitope.

As example for MV-004-mimotopes, original epitope p4372 and the mimotopes p4417, p4418, p4419, and p4420 are depicted in FIG. 16B. 3/4 Mimotope vaccines shown mount detectable immune responses against the original epitope p4372.

As example for MV-001-mimotopes, original epitope p4371 and the mimotopes p4381, p4382, and p4390 are depicted in FIG. 16C. All Mimotope vaccines depicted mount detectable immune responses against the original epitope p4371. A similar phenomenon as described for MV-003 derived mimotopes can be detected analysing cross reactivity against the pyroglutamate-modified form as displayed by p4373. The original epitope p4371-vaccine and all Mimotope vaccines mount relevant titers against p4373. Surprisingly, the mimotopes selected by MV-001, which is specifically binding to p4371 are inducing a immune reaction cross reacting better with the modified form of the original epitope than the original epitope induced immune reaction or the parental antibody. Thus these mimotopes might surprisingly be able to induce but are not necessarily inducing a broader immune reaction than the parental antibody and can be used for a more wide targeting of forms of Aβ.

FIG. 17 shows examples for in vivo characterisations of the immune response elicited by mimotope vaccination against full length Aβ. Surprisingly, the mimotopes selected by using MV-001 and MV-003 induce a cross reaction not only with the truncated or modified short epitopes used to create the antibodies but also induce cross reactivity to full length, non modified forms of Aβ as good as the original sequence or even more efficiently than p4371/p4373. For MV-002 original epitope as well as for the mimotopes identified, no such cross reactivity can be detected demonstrating a transfer of specificity of the antibody to the free N-Terminus of unmodified Aβ11-40/42. Thus the mimotopes presented in this invention constitute optimised vaccine candidates to target a broad spectrum of naturally occurring forms of the Aβ peptides as have been found in the brain of AD patients. The forms include but are not limited to Aβ1-40/42, and N-terminally truncated forms like Aβ3-40/42, Aβ(pE)3-40/42 and unmodified Aβ11-40/42 respectively.

In Table 4 and 5 further examples of the immune response elicited by mimotope vaccination against full length Aβ by using MV-001 and MV-003 derived mimotopes are described.

TABLE 4
In vivo characterisation of mimotopes: MV-001

| Internal Peptide | Detection of |
|---|---|
| number | Aβ/truncated/modified forms |
| p4381 | + |
| p4383 | + |
| p4385 | + |
| p4386 | + |
| p4390 | + |
| p4707 | + |
| p4714 | + |
| p4715 | + |
| p4725 | + |
| p4730 | + |
| p4738 | + |
| p4740 | + |
| p4748 | + |
| p4753 | + |

All peptides listed in Table 4 mount specific immune reactions against full length and/or truncated and modified forms of Aβ or fragments thereof.

TABLE 5
In vivo characterisation of mimotopes: MV-003

| Internal Peptide | Detection of |
|---|---|
| number | Aβ/truncated/modified forms |
| p4395 | + |
| p4396 | + |
| p4397 | + |
| p4399 | + |

All peptides listed in Table 5 mount specific immune reactions against full length and/or truncated and modified forms of Aβ or fragments thereof.

3. Results for MV-002

3.1. Identification of Specific Monoclonal Antibodies (mAB) Directed against N-Terminally Truncated and Modified Forms of Aβ:

FIG. 21 depicts the characterisation of the monoclonal antibody MV-002 (internal name A115; IgG2b) derived from experiment Alz-9 demonstrating specificity for full length Aβ and Aβ fragments truncated at position E11 and H14 and modified at position E11 to pE11.

3.2. Screening with Specific mABs Directed against N-Terminally Truncated and Modified Forms of Aβ:

3.2.1. Phage Display Library Ph.D. 7

3.2.1.1. Screening with Monoclonal Antibody Directed Against p1323

47 Sequences were identified by screening PhD 7 phage display libraries in this screen: Table 1 summarises the peptides identified and their binding capacity as compared to the original epitope.

TABLE 1
mimotopes binding to the parental antibody MV-002

| Internal Peptide | | |
|---|---|---|
| number | Sequence | Binding Capacity |
| p4403 | SHTRLYFC | 1 |
| p4404 | SGEYVFHC | 1 |
| p4413 | SGQLKFPC | 1 |
| p4414 | SGQIWFRC | 1 |
| p4415 | SGEIHFNC | 1 |
| p4666 | GQIWFISC | 1 |
| p4667 | NDAKIVFC | 3 |
| p4668 | GQIIFQSC | 2 |
| p4669 | GQIRFDHC | 3 |
| p4670 | HMRLFFNC | 3 |
| p4671 | GEMWFALC | 3 |
| p4672 | GELQFPPC | 3 |
| p4673 | GELWFPC | 3 |
| p4674 | SHQRLWFC | 3 |
| p4675 | HQKMIFAC | 3 |
| p4676 | GEMQFFIC | 3 |
| p4677 | GELYFRAC | 3 |
| p4678 | GEIRFALC | 3 |
| p4679 | GMIVFPHC | 3 |
| p4680 | GEIWFEGC | 3 |
| p4681 | GEIYFERC | 3 |
| p4682 | AIPLFVMC | 1 |
| p4683 | GDLKFPLC | 3 |
| p4684 | GQILFPVC | 3 |
| p4685 | GELFFPKC | 3 |
| p4686 | GQIMFPRC | 3 |
| p4687 | HMRMYFEC | 3 |
| p4688 | GSLFFWPC | 2 |
| p4689 | GEILFGMC | 3 |
| p4690 | GQLKFPFC | 3 |
| p4691 | KLPLFVMC | 1 |
| p4692 | GTIFFRDC | 1 |
| p4693 | THQRLWFC | 3 |
| p4694 | GQIKFAQC | 3 |
| p4695 | GTLIFHHC | 2 |
| p4696 | GEIRFGSC | 3 |
| p4697 | GQIQFPLC | 3 |
| p4698 | GEIKFDHC | 3 |
| p4699 | GEIQFGAC | 3 |
| p4700 | QLPLFVLC | 1 |
| p4794 | HQKMIFC | 2 |
| p4795 | GELFFEKC | 2 |
| p4796 | GEIRFELC | 2 |
| p4804 | Ac-GEIYFERC | 2 |
| p4805 | SGEIYFERC | 1 |
| p4806 | AGEIYFERC | 1 |
| p4807 | CGEIYFER | 1 |

Legend to Table 1: the binding capacity is coded
by dilution factor of the parental AB. Ac-...
indicates acetylated AA.

| | | OD halfmax |
|---|---|---|
| binding code | | 1:X |
| 0 | no binding | :0 |
| 1 | weak binding | :<40000 |
| 2 | medium binding | :40000-320000 |
| 3 | strong binding | :>320000 |

3.3. In Vitro Characterisation of Mimotopes Identified in Screening Phage Display Libraries with Monoclonal Antibodies Directed against N-Terminally Truncated and Modified Forms of Aβ:

FIGS. 21 and 22 show representative examples for binding and inhibition assays used to characterise mimotopes in vitro. Data obtained are summarised in Tables 1 and 2 respectively.

MV-002 Mimotopes: From the 47 sequences presented 11 sequences inhibited binding of the monoclonal antibody MV-002 in in vitro competition experiments. Additional 36 sequences were identified that did not inhibit binding of monoclonal antibody in in vitro competition experiments but still retained binding capacity to the parental antibody (Table 2). Importantly, as described in FIGS. 23-25, the ability to compete with the original epitope for binding to the parental antibody in vitro was no prerequisite to mount specific immune responses cross reacting with specific peptides in vivo. Thus inhibiting as well as non-inhibiting peptides can be used for inducing immune responses detecting peptides in vivo (for details see: FIGS. 23-25) which can lead to clearance of amyloid peptides from the brain.

TABLE 2
mimotopes identified in this invention giving
positive results in inhibiting assays; MV-002
Mimotopes

| Internal | | |
|---|---|---|
| Peptide | | Inhibition |
| number | Sequence | Capacity |
| p4667 | NDAKIVFC | 1 |
| p4670 | HMRLFFNC | 1 |
| p4673 | GELWFPC | 1 |
| p4674 | SHQRLWFC | 1 |
| p4675 | HQKMIFAC | 2 |
| p4680 | GEIWFEGC | 2 |
| p4681 | GEIYFERC | 2 |
| p4689 | GEILFGMC | 1 |
| p4698 | GEIKFDHC | 2 |
| p4699 | GEIQFGAC | 1 |
| p4794 | HQKMIFC | 1 |

Legend to Table 3: the inhibition capacity is
coded by the following code: Weak inhibition
means more peptide is required to lower AB
binding than with the original epitope;
strong inhibition means similar peptide amounts
are required for mimotope and original
epitope for lowering AB binding. Mimotopes are
compared to the original peptide as standard.
OD at 5 μg peptide used in the assay is used
to calculate the competition capacity
compared to original peptide.

| competition | |
|---|---|
| code | |
| 0 | no inhibition (OD of peptide above |
| | 4, 6 times of original peptide) |
| 1 | Weaker than original epitope (OD |
| | of peptide below 4, 6 times of |
| | original peptide) |
| 2 | strong inhibition (as original |
| | epitope; OD of peptide below 2, 3 |
| | times of original peptide) |

3.4. In Vivo Characterisation of Mimotopes Identified in Screening Phage Display Libraries with a Monoclonal Antibody Directed against Amyloid Beta:

Female C57//b16 mice, 5-6 mice per group, were subcutaneously immunized with 30 μg peptide coupled to KLH. Control groups were administered original epitope-KLH conjugates respectively. As adjuvant alum was used (always 1 mg per mouse). The peptides administered were all able to bind to monoclonal antibodies specifically although some of the peptides did not inhibit the binding of the original epitope to its parental antibody in vitro (in an in vitro inhibition assay). The in vitro ELISA assay to determine the antibody titer was performed with sera of single mice after each vaccination in a two week interval (see FIGS. 25 and 26 respectively). Titers were calculated as OD max/2 in all figures shown. The wells of the ELISA plate were coated with mimotope-BSA conjugate and an irrelevant peptide-BSA conjugate (negative control). The positive control was performed by reaction of the parental antibody with the respective mimotope-BSA conjugate. The detection was performed with anti-mouse IgG. Additionally, recombinant proteins were immobilised on ELISA plates and sera reacted accordingly. FIGS. 23, 24 and 25 show representative examples for assays used to characterise mimotopes in vivo. The results depicted were derived from peptides active in in vitro inhibition assays like p4670, p4675, p4680, and p4681 and a peptide without inhibition capacity, p4403 respectively.

FIG. 23 shows examples for in vivo characterisations of the immune response elicited by mimotope vaccination by analysing the immune response against injected peptide and an irrelevant peptide, containing an unrelated sequence. In the examples shown, the epitope p4377 and the mimotopes p4670, p4675, p4680, p4681 and p4403 elicited immune responses against the injected peptides but failed to induce a relevant unspecific immune response against an unrelated sequence (p1454).

FIG. 24 shows examples for in vivo characterisations of the immune response elicited by mimotope vaccination against the respective original epitope of the parental antibody (p4377) as well as against peptides derived from truncated species of Aβ (p1323 and p4374)and against sAPP alpha.

p4377 and the mimotopes p4670, p4675, p4680, p4681 and p4403 mounted detectable immune responses against the original epitope p4377. A similar phenomenon could be detected analysing cross reactivity against the modified form as displayed by p4374. Interestingly, the original epitope and the mimotope vaccines mounted relevant titers against p4374 the modified form of the original epitope. Surprisingly, the mimotopes seemed to be able to induce but did not necessarily induce a more efficient immune response against p1323 indicating a potential to induce a broader immuno-reactivity as compared to the original Aβ fragment. Additionally, no reactivity was detectable against sAPP alpha.

FIG. 25 shows examples for in vivo characterisations of the immune response elicited by mimotope vaccination against full length Aβ. Surprisingly, the mimotopes selected by using MV-002 induced a cross reaction not only with the truncated or modified short epitopes used to create the antibodies but also induced cross reactivity to full length, non modified forms of Aβ as good as the original sequence or even more efficiently than p4377.

Interestingly competing as well as non competing peptides were able to induce similar immune responses specifically interacting with peptides containing original Aβ sequences. Thus the mimotopes presented in this invention constitute optimised, novel vaccine candidates to target a broad spectrum of naturally occurring forms of the Aβ peptides as have been found in the brain of AD patients. The forms include but are not limited to Aβ1-40/42, and N-terminally truncated forms like Aβ3-40/42, Aβ(pE)3-40/42, unmodified Aβ11-40/42, modified Aβp(E)11-40/42 and Aβ14-40/42 respectively. Importantly, the mimotopes presented also did not induce a cross reactivity to the neoepitopes present in sAPP alpha after cleavage from APP and thus do not interfere with normal sAPP alpha signalling (see FIG. 24 for details).

TABLE 3
Non-Mimotope peptides used

| Internal | |
|---|---|
| Peptide | |
| no. | Sequence |
| p1253 | DAEFRHDSGYC |
| p1323 | CHQKLVFFAED |
| p4374 | p(E)VHHULVFC |
| p4377 | EVHHQKLVFC |
| p1454 | CGLMVGGVV |
| Aβ1-40 | DAEFRHDSGYEVHHQKLVFFAEDVGSNKGAIIGLMVGGVV; |
| | derived from human APP (gi: 112927) |
| Aβ1-42 | DAEFREDSGYEVHHQKLVFFAEDVGSNKGAIIGLMVGGVVIA; |
| | derived from human APP (gi: 112927) |
| sAPPalpha | alpha-Secretase induced cleavage product |
| | derived from human APP (gi: 112927) |

In Table 4 further examples of the immune response elicited by mimotope vaccination against full length Aβ by using MV-002 derived mimotopes are described. All peptides listed in table 4 mount specific immune reactions against full length and/or truncated and modified forms of Aβ or fragments thereof.

TABLE 4
In vivo characterisation of mimotopes: MV-002

| Internal Peptide | Detection of |
|---|---|
| number | Aβ/truncated/modified forms |
| p4403 | + |
| p4404 | + |
| p4413 | + |
| p4414 | + |
| p4415 | + |
| p4670 | + |
| p4673 | + |
| p4675 | + |
| p4680 | + |
| p4681 | + |
| p4693 | + |
| p4696 | + |
| p4698 | + |
| p4699 | + |

### Example 9

### In Vivo Characterisation of Mimotopes for the Efficacy to Reduce AD Like Disease in Transgenic Animals

The Tg2576 AD mouse model was used to study the preclinical efficacy of the mimotope vaccines. This transgenic line is expressing human APP carrying the Swedish double mutation at aa position 670/671 under the control of a hamster prion protein (PrP) promoter which results in overexpression of the protein. It is currently one of the most widely employed models in AD research. The Tg2576 model recapitulates various hallmarks of AD pathology including disease-specific amyloid plaque deposition and astrocytosis. As all other AD model systems available to date, it does not reflect all cardinal neuropathological features of AD.

To assess whether treatment With mimotopes, is capable of preventing cerebral Aβ accumulation, Tg2576 mice were s.c. injected 6 times at monthly intervals with peptide-KLH conjugates adsorbed to ALUM (adjuvant: aluminium hydroxide). or PBS adsorbed to ALUM (referred to as PBS or control) alone. Up to eight weeks after the last immunization, animals were sacrificed, their brains harvested and analyzed for their Aβ load (AD-like pathology). The mice were sacrificed under deep anaesthesia. Subsequently, the brain was isolated, fixed in 4% PFA and dehydrated by graded Ethanol series followed by incubation in Xylene and paraffin embedding. Each paraffin-embedded brain was sectioned at 7 μM using a slicing microtome and sections were mounted on glass slides.

As a method to assay AD-like pathology in Tg2576 animals, the relative area occupied by amyloid deposits in the brain of treated animals was analyzed. This analysis was performed using an automated area recognition programme. To identify the plaques, sections were stained with the monoclonal antibody (mAb) 3A5 (specific for Aβ40/42). Mimotope treated animals were compared to control animals. All animals have been sacrificed at an age of 13.5-14 months. For this analysis 3 slides/animal covering the cortex and hippocampus were selected, stained with mAb 3A5 and subsequently documented using the Mirax-system (Zeiss). For the calculation of the area occupied by amyloid plaques, up to four individual sections per slide were analysed and sections carrying tissue artefacts and aberrant staining intensities have been excluded after inspection of the result pictures.

For the mimotopes derived from MV001 an area analysis using three exemplary candidates was performed: Analysis was performed following repeated vaccination using peptide-KLH conjugate vaccines. The control group showed an average occupation of 0.35% as compared to 0.11%, 0.14% and 0.22% for the mimotope treated animals respectively. This corresponds to a reduction following mimotope treatment of 67% in group 2, a 60% reduction in group 3 and a 36% reduction in group 4 (see FIG. 18).

For the mimotopes of MV002 an area analysis using one exemplary candidate was performed: Analysis was performed following repeated vaccination using peptide-KLH conjugate vaccines. The control group showed an average occupation of 0.35% as compared to 0.24% for the mimotope treated animals respectively. This corresponds to a reduction following mimotope treatment of 31% in group 2.

A similar picture can be detected for the group of MV003 derived mimotopes. Here the example of p4395 is depicted. As described for the MV001 derived mimotopes, an analysis of the area occupied by amyloid plaques following peptide-conjugate vaccination has been performed. The control group showed an average occupation of 0.35% as compared to 0.21% for the mimotope treated animals respectively. This corresponds to a reduction following mimotope treatment of 38% in group 2 (see FIG. 19).

Thus, this set of data clearly indicates a beneficial effect of mimotope vaccine treatment on AD like pathology in transgenic animals.

### Example 10

### In Vivo Characterisation of Mimotopes for the Efficacy to Reduce PD Like Disease in Transgenic Animals (Proof of Concept Analysis)

The double transgenic mouse model (mThy1-APP751 (line TASD41) crossed with mThy1-wt human a-syn (Line TASD 61)) was used to study the preclinical efficacy of AD mimotope vaccines to reduce PD like disease. The model recapitulates various hallmarks of AD and PD pathology including disease-specific amyloid plaque deposition and astrocytosis as well as synuclein aggregation and cell loss.

To assess whether treatment with mimotopes is capable of ameliorating PD like disease, transgenic mice were s.c. injected 6 times at monthly intervals with peptide-KLH conjugates adsorbed to ALUM (adjuvant: aluminium hydroxide) or PBS adsorbed to ALUM (referred to as PBS or control) alone. After the last immunization, animals were sacrificed following guidelines for the humane treatment of animals. Subsequently, the brain was isolated, fixed and sectioned at 40 μM using a vibratome and sections were stored at −20° C. in cryoprotective medium. Sections were immunostained with antibodies against α-synuclein and NeuN (neuronal marker) and imaged with the laser confocal microscope. Digital images were analyzed with the ImageQuant program to assess numbers of α-synuclein aggregates and neurons. Mimotope treated animals were compared to control animals. Results depict an exemplary set of data for a mimotope described in this invention

In order to analyse whether vaccination with AD mimotopes would result in a reduction of PD associated pathology the incidence of neuronal inclusions of α-synuclein in the frontal cortex and the hippocampus was analysed (Lewy body like inclusions). Animals overexpressing APP and α-synuclein in the brain developed pathologic alterations reminiscent of PD. α-synuclein positive neuronal inclusions are depicted in FIG. 27 as spots in neuronal bodies. A quantitative analysis of the inclusions revealed that the levels of accumulation of α-synuclein in the neuronal cell bodies in the neocortex and hippocampus were significantly reduced in the double transgenic mice following AD mimotope vaccination. This reduction amounted to 32.7% in the cortex (p=0.0001) indicating a beneficial effect of AD mimotope vaccination on PD like pathology in this area.

As a second method to assay PD-like pathology in transgenic animals, the number of neurons in the cortex and hippocampus of treated animals by NeuN staining was analyzed.

In this animal model a progressive loss of neurons in the frontal cortex as well as in the hippocampus upon ageing can be detected. Quantification of the neuronal density in the frontal cortex and the hippocampus showed a slight decrease in double transgenic PBS treated mice as compared to non transgenic control animals. This slight reduction indicates neurodegeneration in the strain used for this experiment.

Interestingly, mice treated with an AD mimotope (FIG. 28) showed levels of NeuN positive neurons, which were comparable to controls. Double Tg animals revealed a statistically significant 27% increase (p=0.044) in the hippocampus as compared to the carrier treated controls respectively. In the cortical area, a 28.4% (p=0.0053) increase in the double Tg animals could be observed following AD mimotope treatment. This relative increase as compared to the vehicle treated animals could also be interpreted as an indication of reduced neurodegeneration in successfully treated animals.

Summarizing, this set of data clearly indicates a beneficial effect of AD mimotope vaccine treatment on PD like symptoms in transgenic animals.

1. Compound comprising a peptide for treating, preventing and/or ameliorating motor symptoms of Parkinson's disease, said peptide having a binding capacity to an antibody which is specific for an epitope of the amyloid-beta-peptide (Aβ).

2. Compound according to claim 1, characterised in that said epitope, of the amyloid-beta-peptide is selected from the group consisting of DAEFRH, EFRHDSGY, pEFRHDSGY, EVHHQKL, HQKLVF and HQKLVFFAED.

3. Compound according to claim 1 or 2, characterised in that said peptide has not the amino acid sequence DAEFRH, EFRHDSGY, pEFRHDSGY, EVHHQKL, HQKLVF and HQKLVFFAED.

4. Compound according to any one of claims 1 to 3, characterised in that said peptide comprises the amino acid sequence
X₁X₂X₃X₄X₅X₆X₇, (Formula I)
wherein X₁ is G or an amino acid with a hydroxy group or a negatively charged amino acid, preferably glycine (G), glutamic acid (E), tyrosine (Y), serine (S) or aspartic acid (D),
X₂ is a hydrophobic amino acid or a positively charged amino acid, preferably asparagine (N), isoleucine (I), leucine (L), valine (V), lysine (K), tryptophane (W), arginine (R), tyrosine (Y), phenylalanine (F) or alanine (A),
X₃ is a negatively charged amino acid, preferably aspartic acid (D) or glutamic acid (E),
X₄ is an aromatic amino acid or a hydrophobic amino acid or leucine (L), preferably tyrosine (Y), phenylalanine (F) or leucine (L),
X₅ is histidine (H), lysine (K), tyrosine (Y), phenylalanine (F) or arginine (R), preferably histidine (H), phenylalanine (F) or arginine (R), and
X₆ is not present or serine (S), threonine (T), asparagine (N), glutamine (Q), aspartic acid (D), glutamic acid (E), arginine (R), isoleucine (I), lysine (K), tyrosine (Y), or glycine (G), preferably threonine (T), asparagine (N), aspartic acid (D), arginine (R), isoleucine (I) or glycine (G),
X₇ is not present or any amino acid, preferably proline (P), tyrosine (Y), threonine (T), glutamine (Q), alanine (A), histidine (H) or serine (S),
preferably EIDYHR, ELDYHR, EVDYHR, DIDYHR, DLDYHR, DVDYHR, DI-DYRR, DLDYRR, DVDYRR, DKELRI, DWELRI, YREFFI, YREFRI, YAEFRG, EAEFRG, DYEFRG, ELEFRG, DRELRI, DKELKI, DRELKI, GREFRN, EYEFRG, DWEFRDA, SWEFRT, DKELR, SFEFRG, DAEFRWP, DNEFRSP, GSEFRDY, GAEFRFT, SAEFRTQ, SAEFRAT, SWEFRNP, SWEFRLY, SWELRQA, SVEFRYH, SYEFRHH, SQEFRTP, SSEFRVS, DWEFRD, DAELRY, DWELRQ, SLEFRF, GPEFRW, GKEFRT, AYEFRH, DKE(Nle)R, DKE(Nva)R or DKE(Cha)R.

5. Compound according to any one of claims 1 to 3, characterised in that said peptide comprises the amino acid sequence
X₁RX₂DX₃(X₄)ₙ(X₅)ₘ(X₆)ₒ, (Formula II),
wherein X₁ is isoleucine (I) or valine (V),
X₂ is tryptophan (W) or tyrosine (Y),
X₃ is threonine (T), valine (V), alanine (A), methionine (M), glutamine (Q) or glycine (G),
X₄ is proline (P), alanine (A), tyrosine (Y), serine (S), cysteine (C) or glycine (G),
X₅ is proline (P), leucine (L), glycine (G) or cysteine (C),
X₆ is cysteine (C),
n, m and o are, independently, 0 or 1,
preferably IRWDTP(C), VRWDVYP(C), IRYDAPL(C), IRYDMAG(C), IRWDTSL(C), IRWDQP(C), IRWDG(C) or IRWDGG(C).

6. Compound according to any one of claims 1 to 3, characterised in that said peptide comprises the amino acid sequence
EX₁WHX₂X₃(X₄)ₙ(X₅)ₘ (Formula III),
wherein X₁ is valine (V), arginine (R) or leucine (L),
X₂ is arginine (R) or glutamic acid (E),
X₃ is alanine (A), histidine (H), lysine (K), leucine (L), tyrosine (Y) or glycine (G),
X₄ is proline (P), histidine (H), phenylalanine (F) or glutamine (Q) or Cysteine
X₅ is cysteine (C),
n and m are, independently, 0 or 1,
preferably EVWHRHQ(C), ERWHEKH(C), EVWHRLQ(C), ELWHRYP(C), ELWHRAF(C), ELWHRA(C), EVWHRG(C), EVWHRG(C) and ERWHEK(C), preferably EVWHRHQ(C), ERWHEKH(C), EVWHRLQ(C), ELWHRYP(C) or ELWHRAF(C).

7. Compound according to any one of claims 1 to 3, characterised in that said peptide comprises the amino acid sequence QDFRHY(C), SEFKHG(C), TSFRHG(C), TSVFRH(C), TPFRHT(C), SQFRHY(C), LMFRHN(C), SAFRHH(C), LPFRHG(C), SHFRHG(C), ILFRHG(C), QFKHDL(C), NWFPHP(C), EEFKYS(C), NELRHST(C), GEMRHQP(C), DTYFPRS(C), VELRHSR(C), YSMRHDA(C), AANYFPR(C), SPNQFRH(C), SSSFFPR(C), EDWFFWH(C), SAGSFRH(C), QVMRHHA(C), SEFSHSS(C), QPNLFYH(C), ELFKHHL(C), TLHEFRH(C), ATFRHSP(C), APMYFPH(C), TYFSHSL(C), HEPLFSH(C), SLMRHSS(C), EFLRHTL(C), ATPLFRH(C), QELKRYY(C), THTDFRH(C), LHIPFRH(C), NELFKHF(C), SQYFPRP(C), DEHPFRH(C), MLPFRHG(C), SAMRHSL(C), TPLMFWH(C), LQFKHST(C), ATFRHST(C), TGLMFKH(C), AEFSHWH(C), QSEFKHW(C), AEFMHSV(C), ADHDFRH(C), DGLLFKH(C), IGFRHDS(C), SNSEFRR(C), SELRHST(C), THMEFRR(C), EELRHSV(C), QLFKHSP(C), YEFRHAQ(C), SNFRHSV(C), APIQFRH(C), AYFPHTS(C), NSSELRH(C), TEFRHKA(C), TSTEMWH(C), SQSYFKH(C), (C)SEFKH, SEFKH(C), (C)HEFRH or HEFRH(C).

8. Compound according to any one of claims 1 to 3, characterised in that said peptide comprises the amino acid sequence
(X₁)ₘGX₂X₃X₄FX₅X₆(X₇)ₙ (Formula IV),
wherein X₁ is serine (S), alanine (A) or cysteine (c),
X₂ is serine (S), threonine (T), glutamic acid (E), aspartic acid (D), glutamine (Q) or methionine (M),
X₃ is isoleucine (I), tyrosine (Y), methionine (M) or leucine (L),
X₄ is leucine (L), arginine (R), glutamine (Q), tryptophan (W), valine (V), histidine (H), tyrosine (Y), isoleucine (I), lysine (K) methionine (M) or phenylalanine (F),
X₅ is alanine (A), phenylalanine (F), histidine (H), asparagine (N), arginine (R), glutamic acid (E), isoleucine (I), glutamine (Q), aspartic acid (D), proline (P) or tryptophane (W), glycine (G)
X₆ is any amino acid residue,
X₇ is cysteine (C),
m and n are, independently, 0 or 1,
preferably SGEYVFH(C), SGQLKFP(C), SGQIWFR(C), SGEIHFN(C), GQIWFIS(C), GQIIFQS(C), GQIRFDH(C), GEMWFAL(C), GELQFPP(C), GELWFP(C), GEMQFFI(C), GELYFRA(C), GEIRFAL(C), GMIVFPH(C), GEIWFEG(C), GDLKFPL(C), GQILFPV(C), GELFFPK(C), GQIMFPR(C), GSLFFWP(C), GEILFGM(C), GQLKFPF(C), GTIFFRD(C), GQIKFAQ(C), GTLIFHH(C), GEIRFGS(C), GQIQFPL(C), GEIKFDH(C), GEIQFGA(C), GELFFEK(C), GEIRFEL(C), GEIYFER(C), SGEIYFER(C), AGEIYFER(C) or (C)GEIYFER.

9. Compound according to any one of claims 1 to 3, characterised in that said peptide comprises the amino acid sequence
(X₁)ₘHX₂X₃X₄X₅FX₆(X₇)ₙ (Formula V),
wherein X₁ is serine (S), threonine (T) or cysteine (C),
X₂ is glutamine (Q), threonine (T) or methionine (M),
X₃ is lysine (K) or arginine (R),
X₄ is leucine (L), methionine (M),
X₅ is tryptophane (W), tyrosine (Y), phenylalanine (F) or isoleucine (I),
X₆ is asparagine (N), glutamic acid (E), alanine (A) or cysteine (C),
X₇ is cysteine (C),
n and m are, independently, 0 or 1,
preferably SHTRLYF(C), HMRLFFN(C), SHQRLWF(C), HQKMIFA(C), HMRMYFE(C), THQRLWF(C) or HQKMIF(C).

10. Compound according to any one of claims 1 to 3, characterised in that said peptide comprises the amino acid sequence AIPLFVM(C), KLPLFVM(C), QLPLFVL(C) or NDAKIVF(C).

11. Compound according to any one of claims 1 to 10, characterised in that the compound is a polypeptide and comprises 4 to 30 amino acid residues.

12. Compound according to any one of claims 1 to 11, characterised in that the compound is coupled to a pharmaceutically acceptable carrier, preferably KLH (Keyhole Limpet Hemocyanin).

13. Compound according to any one of claims 1 to 12, characterised in that the compound is formulated for subcutaneous, intradermal or intramuscular administration.

14. Compound according to any one of claims 1 to 13, characterised in that the compound is formulated with an adjuvant, preferably aluminium hydroxide.

15. Compound according to any one of claims 1 to 14, characterised in that the compound is contained in the medicament in an amount of from 0.1 ng to 10 mg, preferably 10 ng to 1 mg, in particular 100 ng to 10 μg.

16. Compound according to any one of claims 1 to 15, characterised in that the motor symptoms of Parkinson's disease are selected from the group consisting of resting tremor, Bradykinesia, rigidity, postural instability, stooped posture, dystonia, fatigue, impaired fine motor dexterity and motor coordination, impaired gross motor coordination, poverty of movement (decreased arm swing), akathisia, speech problems, loss of facial expression, micrographia, difficulty swallowing, sexual dysfunction and drooling.

17. Use of a compound according to any one of claims 1 to 12 for the manufacture of a medicament for treating, preventing and/or ameliorating motor symptoms of Parkinson's disease.
